(19) United States
(12) Patent Application Publication
(10) Pub. No.: US 2023/0019058 A1
(43) Pub. Date: Jan. 19, 2023

(54) METHOD FOR THE TREATMENT OF VIRAL INFECTIONS

(71) Applicant: Softhale NV, Diepenbeek (BE)

(72) Inventors: Juergen Rawert, Koeln (DE); Jan-Torsten Tews, Munich (DE)

(21) Appl. No.: 17/931,139

(22) Filed: Sep. 12, 2022

Related U.S. Application Data
(63) Continuation-in-part of application No. PCT/EP2021/056327, filed on Mar. 12, 2021.
Application No. PCT/EP2021/056327 is a continuation of application No. 16/907,336, filed on Jun. 22, 2020, now abandoned.
(60) Provisional application No. 62/989,414, filed on Mar. 13, 2020.

(51) Int. Cl. A61K 31/58; A61M 11/00; A61K 9/00

(52) U.S. Cl. CPC A61K 31/58; A61M 11/007; A61K 9/0078; A61M 2250/00

(57) ABSTRACT

The present invention provides for a method for the treatment of a respiratory disease, disorder, or condition resulting from a viral infection in a subject, such as a pulmonary viral infection, the method comprising the step of administering to said subject a medically active liquid in nebulized form by inhalation, wherein the medically active liquid comprises an inhalable corticosteroid (ICS) and wherein the medically active liquid is administered in nebulized form using an inhalation device.

### CROSS REFERENCE TO RELATED APPLICATIONS

The present application is a continuation-in-part application of PCT International Application No. PCT/EP2021/056327, filed on Mar. 12, 2021, which claims priority to and the benefit of U.S. application Ser. No. 16/907,336, filed on Jun. 22, 2020, and U.S. Provisional Application No. 62/989,414, filed on Mar. 13, 2020, all of which are hereby incorporated herein by reference in their entireties.

### BACKGROUND OF THE INVENTION

The present invention relates to the field of methods for the treatment and compositions for use in the treatment of viral infections, more specifically pulmonary viral infections such as infections by coronavirus. Furthermore, the present invention relates to the field of inhalation devices or the administration of medically active liquids for inhalation therapy. More specifically, the present invention relates to the administration of a medically active liquid comprising an inhalable corticosteroid (ICS) by inhalation.

Nebulizers or other aerosol generators for liquids are known from the art since a long time ago. Amongst others, such devices are used in medical science and therapy. There, they serve as inhalation devices for the application of active ingredients in the form of aerosols, i.e., small liquid droplets embedded in a gas. Such an inhalation device is known e.g., from document EP 0 627 230 B1. Essential components of this inhalation device are a reservoir in which the liquid that is to be aerosolized is contained; a pumping device for generation of a pressure being sufficiently high for nebulizing; as well as an atomizing device in the form of a nozzle. By means of the pumping device, the liquid is drawn in a discrete amount, i.e., not continuously, from the reservoir, and fed to the nozzle. The pumping device works without propellant and generates pressure mechanically.

WO 2018/19770 A1 discloses a soft mist inhalation device (SMI) having an impingement type nozzle which has proven to be useful for the effective administration of pharmaceutically active liquids especially in cases in which the medically active liquid or, more specifically, the pharmaceutically active compound or ingredient contained therein has to be administered to the lungs of the patient or other subject in need thereof.

S. Matsuyama et al. have recently reported that the inhaled corticosteroid ciclesonide blocks coronavirus RNA replication by targeting the viral protein NSP15 (Non-Structural Protein 15) (doi.org/10.1101/2020.0311.987016). The authors report that steroid compounds, which are expected to have dual functions in blocking host inflammation and coronavirus (e.g., MERS-CoV) replication, were screened from a chemical library. Within this library, ciclesonide, as an inhalable corticosteroid, suppressed human coronavirus replication in cultured cells, but did not suppress replication of respiratory syncytial virus or influenza virus. The effective concentration of ciclesonide to block SARS-CoV-2 (the cause of COVID-19) replication (EC90) was 6.3 μM. After the eleventh consecutive coronavirus passage in the presence of ciclesonide, a resistant mutation was generated, which resulted in an amino acid substitution (A25V) in NSP15, as identified using reverse genetics. A recombinant virus with the mutation was also resistant to ciclesonide suppression of viral replication. These observations suggest that the effect of ciclesonide was specific to coronavirus, and, furthermore, that ciclesonide might be a candidate drug for the treatment of patients suffering from human respiratory coronaviruses, including the SARS (severe acute respiratory syndrome) viruses, such as MERS and COVID-19.

It is thus an object of the present invention to provide a method for the effective administration of inhaled corticosteroids (ICS), especially ciclesonide in a convenient, effective and patient friendly manner that allows for the treatment of patients suffering from viral pulmonary infections such as COVID-19, especially in cases in which a large number of subjects has to be treated effectively. Further objects of the invention will be clear on the basis of the following description of the invention, examples and claims.

### SUMMARY OF THE INVENTION

In a first aspect, the present invention provides for a medically active liquid comprising an inhalable corticosteroid (ICS) for use in the treatment of a respiratory disease, disorder or condition resulting from a viral infection in a subject, wherein the medically active liquid is administered to the subject in nebulized form by inhalation using an inhalation device.

In a second aspect, the invention relates to a method for the treatment of a respiratory disease, disorder, or condition resulting from a viral infection in a subject, the method comprising the step of administering to said subject a medically active liquid in nebulized form by inhalation, wherein the medically active liquid comprises an inhalable corticosteroid (ICS) and wherein the medically active liquid is administered in nebulized form using an inhalation device.

In a further aspect, the invention relates to a use of a medically active liquid comprising an inhalable corticosteroid (ICS) for the treatment of a respiratory disease, disorder or condition resulting from a viral infection in a subject, wherein the medically active liquid is used by inhalation of the medically active liquid in nebulized form, wherein the medically active liquid in nebulized form is generated by nebulization using an inhalation device.

In yet a further aspect, the invention relates to a use of an inhalable corticosteroid (ICS) in the manufacture of a medically active liquid for the treatment of a respiratory disease, disorder or condition resulting from a viral infection in a subject, wherein the medically active liquid is administered to the subject in nebulized form by inhalation, and wherein the medically active liquid in nebulized form is generated using an inhalation device.

In yet a further aspect, the present invention provides for a kit, the kit comprising
a medically active liquid comprising an inhalable corticosteroid (ICS) for the treatment of a respiratory disease, disorder or condition resulting from a viral infection in a subject, preferably ciclesonide, wherein the medically active liquid is administered to the subject in nebulized form by inhalation; and
an inhalation device, preferably a hand-held inhalation device, such as a soft-mist inhaler.

### BRIEF DESCRIPTION OF THE DRAWINGS

FIG. 1 depicts solubilities of selected inhalable corticosteroids in selected solvent systems;

FIG. 2 shows the average particle (drop) size distribution, entire spray duration, for ciclesonide in 70:30 ethanol-water (w/w %) via soft-mist inhaler nebulization as measured by laser diffraction;

FIG. 3 shows an embodiment of an inhalation device that may be used in the methods, uses and medically active liquids for use according to the present invention prior to its first use;

FIG. 4 shows an inhalation device similar to the one of FIG. 3, but without an outlet valve;

FIG. 5 shows the embodiment of FIG. 3, with a filled pumping chamber;

FIG. 6 shows the situation during the first actuation of the inhalation device of FIG. 3;

FIG. 7 shows the situation at the end of the first actuation;

FIG. 8 shows the situation after re-filling the pumping chamber;

FIG. 9 shows the average particle (drop) size distribution, entire spray duration, for ciclesonide in 70:30 ethanol-water (w/w %) in the presence of Tween 80 via soft-mist inhaler nebulization as measured by laser diffraction;

FIG. 10 shows the average particle (drop) size distribution, entire spray duration, for ciclesonide in 70:30 ethanol-water (w/w %) in the presence of PEG300 via soft-mist inhaler nebulization as measured by laser diffraction;

FIG. 11 shows the average particle (drop) size distribution, entire spray duration, for ciclesonide in 65:35 and 70:30 ethanol-water (w/w %) mixtures and 65:35 and 70:30 ethanol-water (w/w %) mixtures in the presence of PEG400 via soft-mist inhaler nebulization as measured by laser diffraction;

FIG. 12 shows the average particle (drop) size distribution, entire spray duration, for ciclesonide in 70:30 and 80:20 ethanol-water (w/w %) mixtures and 70:30 and 80:20 ethanol-water (w/w %) mixtures in the presence of PEG400 via soft-mist inhaler nebulization as measured by laser diffraction;

FIG. 13 shows the average particle (drop) size distribution, entire spray duration, for ciclesonide in 65:35, 70:30, and 80:20 ethanol-water (w/w %) mixtures; and

FIG. 14 shows the average particle (drop) size distribution, entire spray duration, for ciclesonide in 65:35, 70:30, and 80:20 ethanol-water (w/w %) mixtures in the presence of PEG400.

### DETAILED DESCRIPTION OF THE INVENTION

In a first aspect, the present invention provides for a medically active liquid comprising an inhalable corticosteroid (ICS) for use in the treatment of a respiratory disease, disorder or condition resulting from a viral infection in a subject, wherein the medically active liquid is administered to the subject in nebulized form by inhalation using an inhalation device.

In a further aspect, the present invention provides for a method, for the treatment of a respiratory disease, disorder or condition resulting from a viral infection in a subject, the method comprising the step of administering to said subject a medically active liquid in nebulized form by inhalation, wherein the medically active liquid comprises an inhalable corticosteroid (ICS) and wherein the medically active liquid is administered in nebulized form using an inhalation device.

In yet a further aspect, the present invention relates to a use of an inhalable corticosteroid (ICS) in the manufacture of a medically active liquid for the treatment of a respiratory disease, disorder or condition resulting from a viral infection in a subject, wherein the medically active liquid is administered to the subject in nebulized form by inhalation, and wherein the medically active liquid in nebulized form is generated using an inhalation device.

In yet a further aspect, the invention relates to a use of a medically active liquid comprising an inhalable corticosteroid (ICS) for the treatment of a respiratory disease, disorder or condition resulting from a viral infection in a subject, wherein the medically active liquid is used by inhalation of the medically active liquid in nebulized form, wherein the medically active liquid in nebulized form is generated by nebulization using an inhalation device.

More specifically, the present invention further provides for a method for the treatment, a medically active liquid for the use or the use thereof in the treatment of a pulmonary viral infection in a subject, wherein the medically active liquid comprises an inhalable corticosteroid (ICS) and wherein the medically active liquid is administered in nebulized form by inhalation using an inhalation device.

Introductorily, some definitions of terms are given which are used throughout the description and claims. The definitions should be used to determine the meaning of the respective expressions unless the context requires a different meaning.

The term “about” or the like in connection with an attribute or value includes the exact attribute or precise value, as well as any attribute or value typically considered to fall within the normal or accepted variability associated with the technical field, and methods of measuring or determining said attribute or value.

“Atomization” and “nebulization” in the context of inhalers means the generation of fine, inhalable droplets of a liquid. The typical dimensions of atomized droplets are in the range of several microns.

An “aerosol” is a dispersion of a solid or liquid phase in a gas phase. The dispersed phase, also termed the discontinuous phase, is comprised of multiple solid or liquid particles. The aerosol generated by the inhalation device of the invention is a dispersion of a liquid phase in the form of inhalable liquid droplets in a gas phase which is typically air. The dispersed liquid phase may optionally comprise solid particles dispersed in the liquid.

The term “comprising,” and related terms “comprise” or “comprises” would be understood as meaning that features additional to the features prefaced by the term may be present. Conversely, the term “consists,” and related terms would be understood as meaning that no other features, other than those prefaced by the term are present, and if present, only in trace or residual amounts such as to confer no technical advantage or relevance in respect of the object of the invention.

The term “medically active liquid” as used herein means a pharmaceutically acceptable liquid comprising at least one medically active compound.

As used herein, the term “medically active” refers to a compound which has pharmacologically activity which improves respiratory function and/or has an anti-viral effect (e.g., inhibiting the viral life cycle).

The term “effective amount” as used herein refers to the administration of an amount of the relevant compound or composition sufficient to prevent the occurrence of symptoms of the condition being treated, or to bring about a halt in the worsening of symptoms or to treat and alleviate or at least reduce the severity of the symptoms. The effective amount will vary in a manner which would be understood by a person of skill in the art with patient age, sex, weight etc. Further definitions are provided in the subsequent description.

For the avoidance of doubt, it should be noted that all embodiments and features of the present invention as well as combinations thereof as described below regardless of being referred to as “specific”, “particular”, “preferred”, “advantageous” or in any other way may refer to all aspects of the present invention as summarized above and as additionally described below.

The medically active liquid to be administered according to the present invention comprises an inhalable corticosteroid (ICS) which may be selected from a broad variety of corticosteroids that are suitable for inhalation or inhalative administration to a subject, more specifically to a warm-blooded animal or human, especially to human in need thereof. In specific embodiments, a “subject” according to the present invention may be a human, more specifically a post-pubertal human and even more specifically a human of at least 12, or of at least 14, or at least 16 or at least 18 years of age.

In specific embodiments, the inhalable corticosteroid comprised by the medically active liquid according to the present invention may, for example, be selected from the group of inhalable corticosteroids consisting of prednisolone (11,17-Dihydroxy-17-(2-hydroxyacetyl)-10,13-dimethyl-6,7,8,9,10,11,12,13,14,15,16,17-dodecahydrocyclopenta[a] phenanthren-3-one; CAS Nr. [50-24-8]), prednisone (CAS Nr. [53-03-2]), butixocort (e.g., tixocortol butyrate (11β,17α-Dihydroxy-21-mercaptopregn-4-ene-3,20-dione 17-butyrate; CAS Nr. [120815-74-9]) or butixocort propionate, CAS Nr. [98449-05-9]), flunisolide (6α-Fluoro-11β,16α,17,21-tetrahydroxypregna-1,4-diene-3,20-dione acetone cyclic 16,17-acetal; CAS Nr. [3385-03-3]), beclomethasone (beclomethasone dipropionate; CAS Nr. [5534-09-8]), triamcinolone (CAS Nr. [124-94-7]), budesonide (CAS Nr. [51333-22-3]), fluticasone (fluticasone furoate, CAS Nr. [397864-44-7] or fluticasone proprionate, CAS Nr. [80474-14-2]), mometasone (CAS Nr. [105102-22-5]), mometasone furoate (9α,21-Dichlor-11β,17α-dihydroxy-16α-methyl-1,4-pregnadien-3,20-dion-17-(2-furoat), CAS Nr. [83919-23-7]), ciclesonide, rofleponide (CAS Nr. 144459-70-1), dexamethasone ((11β,16α)-9-Fluor-11,17,21-trihydroxy-16-methylpregna-1,4-dien-3,20-dion, 9α-Fluor-16α-methyl-11β,17α,21-trihydroxy-1,4-pregnadien-3,20-dion, CAS Nr. [50-02-2]), etiprednol (e.g. etiprednol dichloroacetate, CAS Nr. [199331-40-3]), deflazacort (CAS Nr. [14484-47-0]), loteprednol (loteprednole etabonate; CAS Nr. [82034-46-6]), RPR-106541 ((20R-16alpha,17alpha-[butylidenebis(oxy)]-6alpha, 9alpha-difluoro-11beta-hydroxy-17beta-(methylthio)androsta-4-en-3-one) and NS-126 (9-fluoro-11beta,17,21-trihydroxy-16alpha-methylpregna-1,4-diene-3,20-dione 21-cyclohexanecarboxylate 17-cyclopropanecarboxylate).

More specifically, the inhalable corticosteroid comprised by the medically active liquid according to the present invention may be selected from the group of corticosteroids consisting of beclomethasone, budesonide, dexamethasone, fluticasone, mometasone (furoate) and ciclesonide.

In especially preferred embodiments, the medically active liquid comprises ciclesonide as the inhalable corticosteroid. As used herein, the term “ciclesonide” refers to a compound having the International Union of Pure and Applied Chemistry (IUPAC) name (11β, 16α)-16, 17-[[(R)-cyclohexylmethylene]bis(oxy)]-11-hydroxy-21-(2-methyl-1-oxopropoxy)-pregna-1,4-diene-3,20-dione and the Chemical Abstracts Service (CAS) number [126544-47-6]. Ciclesonide has a molecular weight of 540.697 g/mol and the following structure:

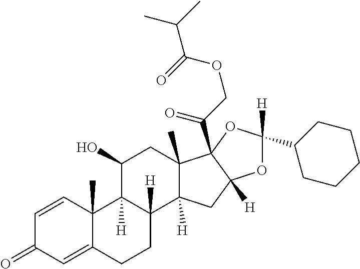

In specific embodiments, the term “ciclesonide” also includes any pharmaceutically acceptable salt, hydrate, solvate or prodrug thereof. In specific embodiments, the medically active liquid comprises ciclesonide in the form of its R-enantiomer.

It should be noted however, that the inhalable corticosteroids as described above may be administered as the sole inhalable corticosteroid or may be comprised by the medically active liquid in the form of a mixture of two or more of the inhalable corticosteroids as described above which may be comprised by the medically active liquid according to the present invention.

In further specific embodiments the chosen inhalable corticosteroid or mixture of two or more inhalable corticosteroids may be administered to the respiratory tract, specifically to the lower respiratory tract and even more specifically to the lungs of the subject in need thereof.

The medically active liquid for use, method and/or use according to the present invention allows for the treatment of a respiratory disease, disorder, or condition resulting from a viral infection in a subject. Additionally, the medically active liquid for use, method and/or use according to the present invention allow for the treatment of the pulmonary viral infection in a patient or subject.

In specific embodiments, the respiratory disease, disorder or condition is an inflammatory disease, disorder or condition, optionally caused or initiated by a pathogen, such by a viral infection as outlined in further detail below.

Viral infections such as pulmonary viral infections that may be treated according to the present invention may be selected from a broad variety of viral infections including coronavirus, influenza virus, rhinovirus, and adenovirus, such as SARS viruses, MERS viruses, H1N1 influenza, and Avian Flu H5N1, specifically severe acute respiratory syndrome viruses (SARS) such as severe acute respiratory syndrome coronaviruses (SARS-CoV or SARS-CoV-2), Middle East respiratory syndrome viruses such as Middle East respiratory syndrome coronaviruses (MERS-CoV). In specific embodiments, however, the pulmonary viral infection to be treated according to the present invention is an infection by a coronavirus. In some embodiments, the pulmonary viral infection is a lower respiratory tract infection such as an infection of the lungs (e.g., a pneumonia).

In further specific embodiments, the viral infection, e.g., pulmonary viral infection, to be treated according to the present invention is a SARS-CoV-2 virus infection. Such SARS-CoV-2 virus infection it is believed to be the cause of the pandemic disease COVID-19. Accordingly, in specific embodiments, the present invention allows for the treatment of pulmonary viral infections in a subject or patient diagnosed with COVID-19.

In further specific embodiments as mentioned above, the disease, disorder or condition to be treated according to the present invention is a lower respiratory tract infection, affecting at least a part of the lower respiratory tract of a subject, specifically a human, such as one or both lungs of a subject or patient (e.g., a pneumonia). According to these embodiments, the respiratory disease, disorder or condition may be a pulmonary disease, disorder or condition, whereas the term “pulmonary” means that such disease affects or is associated with one or both lungs of a subject or patient. In some embodiments, the respiratory disease, disorder or condition or viral disease, disorder or condition may be induced by or result from a viral infection.

The inhalable corticosteroid (ICS), preferably selected from the group consisting of beclomethasone, budesonide, dexamethasone, fluticasone, mometasone (furoate) and ciclesonide, preferably ciclesonide, comprised by the medically active liquid of the present invention may be administered to the subject in need thereof or patient in an effective amount, for example in an amount of about 10 μg (micrograms, mcg) to about 3,000 μg (three thousand mcg) per day or from about 100 μg (mcg) to about 2,000 μg (mcg) per day or from about 200 μg (mcg) to about 1,000 μg (mcg) per day.

In further specific embodiments, the medically active liquid according to the present invention may comprise in addition to the inhalable corticosteroid or the combination of two or more different inhalable corticosteroids one or more further pharmaceutically active compounds (APIs) that are suitable for inhalative administration.

In other specific embodiments, however, the medically active liquid according to the present invention comprises the selected inhalable corticosteroid or the combination of two or more different inhalable corticosteroids as the only medically or pharmaceutically active compound(s). In even more specific embodiments, the medically active liquid according to the present invent comprise the selected inhalable corticosteroid, preferably selected from the group consisting of beclomethasone, budesonide, fluticasone, dexamethasone, mometasone (furoate) and ciclesonide, preferably ciclesonide, as the only medically or pharmaceutically active compound.

The medically active liquid or, in other words, liquid pharmaceutical composition to be administered or for use according to the invention is preferably formulated as a composition that is suitable, and adapted for inhalative use or administration, or in other words is a composition that may be nebulized or atomized for inhalation and that is physiologically acceptable for inhalation by a subject.

The medically active liquid or liquid pharmaceutical composition to be administered by inhalation according to the invention may be in the form of a dispersion, for example a suspension with a liquid continuous phase and a solid dispersed phase or in the form of an emulsion with a liquid continuous phase and a liquid dispersed phase or in the form of a solution.

In further embodiments, the medically active liquid or pharmaceutical composition according to the present invention may comprise, optionally, one or more physiologically acceptable excipients, which are suitable for inhalative use. Excipients which may be used in the medically active liquid or liquid composition include, but are not limited to, one or more buffering agents to regulate or control pH of the solution, chelating agents, salts such as sodium chloride, taste-masking agents, surfactants, lipids, antioxidants, and co-solvents, which may be used to enhance or improve solubility.

Suitable excipients are known to the skilled person and are described, e.g., in standard pharmacopoeias such as U.S.P. or Ph. Eur., or in the Handbook of Pharmaceutical Excipients, 6th ed. Rowe et al, Eds.; The Pharmaceutical Press and the American Pharmaceutical Association: 2009.

Exemplary compounds suitable as buffers for the adjustment of the pH of the present medically active liquid comprise, for example, sodium dihydrogen phosphate dihydrate and/or disodium hydrogen phosphate dodecahydrate, sodium hydroxide solution, basic salts of sodium, calcium or magnesium such as, for example, citrates, phosphates, acetates, tartrates, lactates etc., amino acids, acidic salts such as hydrogen phosphates or dihydrogen phosphates, especially those of sodium, moreover, organic and inorganic acids such as, for example, hydrochloric acid, sulphuric acid, phosphoric acid, citric acid, cromoglycinic acid, acetic acid, lactic acid, tartaric acid, succinic acid, fumaric acid, lysine, methionine, acidic hydrogen phosphates of sodium or potassium, etc., and further buffer systems as described above. In further specific embodiments, the medically active liquid to be nebulized and administered according to the present invention may comprise one or more further excipients which are selected from chelating agents, for example, disodium edetate dihydrate, calcium sodium EDTA, preferably disodium edetate dihydrate.

In some embodiments, the pH of the medically active liquid is from about 3.0 to about 6.0. In other embodiments, the pH of the medically active liquid is from about 4.0 to about 5.0. In yet other embodiments, the pH of the medically active liquid is from about 4.25 to about 4.75, such as about 4.5. In particular embodiments, the pH is adjusted with hydrochloric acid.

In yet further specific embodiments, the medically active liquid to be nebulized and administered according to the present invention may comprise one or more preservatives and/or antioxidants. Suitable preservatives comprise but are not limited to benzalkonium chloride (BAC), parabens such as methylparaben, ethylparaben, propylparaben, sodium benzoate, sorbic acid and salts thereof. In specific embodiments, the medically active liquid to be nebulized and administered according to the present invention comprises benzalkonium chloride as a preservative. Suitable antioxidants comprise but are not limited to butylated hydroxytoluene (BHT), vitamin A, vitamin E, vitamin C, retinyl palmitate and others.

Further excipients that may be included in the medically active liquid comprising an inhalable corticosteroid, preferably beclomethasone, budesonide, dexamethasone, fluticasone, mometasone (furoate) and/or ciclesonide, especially ciclesonide, to be administered according to the present invention comprise, but are not limited to phoshatidylcholines, such as dilauroylphosphatidylcholine (DLPC), dipalmitoylphosphatidylcholine (DPPC), distearoylphosphatidyl glycerol (DTPA), diethylene triamine pentaacetic acid, hydrogenated soy phosphatidylcholine (HSPC), multilamellar vesicles, and soy phosphatidylcholine (SPC).

In particular embodiments, the medically active liquid does not comprise a surfactant. In other embodiments, the medically active liquid does not comprise a non-ionic surfactant. In other embodiments, the medically active liquid does not comprise a polysorbate surfactant. In yet other embodiments, the medically active liquid does not comprise polysorbate 80 (Tween 80).

Excipients which may be featured in the medically active liquid include, but are not limited to, one or more buffering agents to regulate or control pH of the solution, salts, taste-masking agents, surfactants, lipids, antioxidants, and co-solvents, which may be used to enhance or improve solubility; for example, water, alcohols, specifically alcohols with 2 to 4, or preferably 2 or 3 carbon atoms, such as ethanol, propanol or iso-propanol or a glycol.

In specific embodiments, the medically active liquid to be nebulized and administered according to the present invention comprises the inhalable corticosteroid and optionally the further pharmaceutically active ingredients or excipients dissolved in an alcoholic or aqueous liquid vehicle or solvent. In preferred embodiments, such liquid vehicle or solvent comprises water and/or ethanol, preferably ethanol. In further specific embodiments, such liquid vehicle or solvent comprises or preferably consists of ethanol or a mixture of ethanol and water, wherein ethanol may be comprised in an amount of at least about 50 wt.-%, or at least about 60 wt.-% or at least about 70 wt.-% or even more and water in a corresponding amount of up to about 50 wt.-%, or up to about 40 wt.-% or up to about 30 wt.-% or less. In specific embodiments, the liquid vehicle or solvent comprises or consists of ethanol in an amount of about 60 to about 80 wt.-%, such as about 70 wt.-%, and water in an amount of about 40 to about 20 wt.-%, such as about 30 wt.-%.

In specific embodiments, the resulting particle size of the nebulized medically active liquid may be controlled by the addition of additives, such as, for example, water and/or a polyether compound as discussed in more detail in the Examples, below. Although particles below 5 μm allows for good inhalability of the nebulized medically active liquid into the lungs of a subject, smaller particle sizes may result in more of the formulation being exhaled instead of staying in the lungs. Therefore, a shift to larger particle sizes may also be desirable. Additives that were hypothesized to shift average particle size distributions to larger values included water, surfactants, and polyethers such as polyethylene glycol. However, the inventors found that the addition of a non-ionic surfactant, such as a polysorbate (e.g., polysorbate 80), unexpectedly had little impact to increase particle size of the nebulized medically active liquid relative to pure ethanol mixtures. However, increasing the water concentration from 80:20 to 70:30 and to 65:35 (w/w %) increases the particle size relative to pure ethanol mixtures as desired. The addition of a polyether compound, such as a polyethylene glycol, i.e., a polyethylene glycol having an average molecular weight of approximately 300 Daltons such as polyethylene glycol 300 or approximately 400 Daltons such as polyethylene glycol 400, was expected to behave similarly to water and increase the particle size with increasing concentrations. However, the inventors unexpectedly found that the addition of polyethylene glycol to the 65:35, 70:30, and 80:20 (w/w %) ethanol:water mixtures does not follow the same trend as ethanol:water alone and results in a maximum particle size at the 70:30 (w/w %) ethanol:water concentration and lower particle sizes at the 65:35 and 80:20 (w/w %) ethanol:water concentrations. The 70:30 and 80:20 (w/w %) ethanol:water concentrations in the presence of polyethylene glycol, in particular polyethylene glycol 300 or 400, especially polyethylene glycol 400 or, more generally a polyethylene glycol having an average molecular weight selected within the range of from about 300 to about 400, gave particularly desirable particle size distributions of 4.83 and 5.67 μm at the Dv50, respectively. The terms “Dv10, Dv50, and Dv90” refer to the size point below which there is 10%, 50%, and 90% of the volume of the sample, respectively. In other words, the Dv10 is the size point below which there is 10% of the volume of the sample, the Dv50 is the size point below which there is 50% of the volume of the sample, and the Dv90 is the size point below which there is 90% of the volume of the sample. Therefore, ethanol:water concentrations in the range from about 70:30 (w/w %) ethanol:water to about 80:20 (w/w % ethanol:water) are particularly desirable. In some particular embodiments, the ethanol:water concentration is about 70:30 (w/w %) ethanol:water.

In specific embodiments, the medically active liquid further comprises a polyether compound. In some embodiments, the polyether compound is a polyethylene glycol (CAS-Nr. 25322-68-3). As used herein the term “polyethylene glycol” refers to a polyether compound having the structure H—(O—CH₂—CH₂)ₙ—OH and also referred to as “poly(oxyethylene)” and “poly(ethylene oxide)”. In some embodiments, the polyethylene glycol is polyethylene glycol 300 (PEG300) or polyethylene glycol 400 (PEG400). In some specific embodiments, the polyethylene glycol is polyethylene glycol 300 (PEG300). In further specific embodiments, the polyethylene glycol is polyethylene glycol 400 (PEG400).

In further specific embodiments, the concentration of the polyethylene glycol in the medically active liquid, such as PEG300 or PEG400, is from about 150 mg/mL to about 250 mg/mL with regard to the final medically active liquid to be nebulized. In other embodiments, the concentration is from about 175 mg/mL to about 225 mg/mL. In yet other embodiments, the concentration is about 150 mg/mL, about 155 mg/mL, about 160 mg/mL, about 165 mg/mL, about 170 mg/mL, about 175 mg/mL, about 180 mg/mL, about 185 mg/mL, about 190 mg/mL, about 195 mg/mL, about 200 mg/mL, about 205 mg/mL, about 210 mg/mL, about 215 mg/mL, about 220 mg/mL, about 225 mg/mL, about 230 mg/mL, about 235 mg/mL, about 240 mg/mL, about 245 mg/mL, or about 250 mg/mL. In some particular embodiments, the concentration is about 200 mg/mL.

In further specific embodiments, the inhalable corticosteroid is ciclesonide, the medically active liquid comprises a mixture of ethanol and water and further comprises a polyether compound, and the pH of the medically active liquid is from about 4.0 to about 5.0. In further specific embodiments, the mixture of ethanol and water is an about 70:30 (w/w %) ethanol:water mixture, an about 80:20 (w/w %) ethanol:water mixture, or an ethanol:water mixture in the range from about 70:30 (w/w %) ethanol:water to about 80:20 (w/w %) ethanol:water. In further specific embodiments, the mixture of ethanol and water is an about 70:30 (w/w %) ethanol:water mixture. In yet further specific embodiments, the medically active liquid does not comprise a non-ionic surfactant, such as a polysorbate surfactant, for example, polysorbate 80.

In further specific embodiments, the inhalable corticosteroid is ciclesonide, the medically active liquid comprises a mixture of ethanol and water and further comprises a polyethylene glycol, and the pH of the medically active liquid is from about 4.0 to about 5.0. In further specific embodiments, the mixture of ethanol and water is an about 70:30 (w/w %) ethanol:water mixture, an about 80:20 (w/w %) ethanol:water mixture, or an ethanol:water mixture in the range from about 70:30 (w/w %) ethanol:water to about 80:20 (w/w %) ethanol:water. In further specific embodiments, the mixture of ethanol and water is an about 70:30 (w/w %) ethanol:water mixture. In yet further specific embodiments, the medically active liquid does not comprise a non-ionic surfactant, such as a polysorbate surfactant, for example, polysorbate 80.

In further specific embodiments, the inhalable corticosteroid is ciclesonide, the medically active liquid comprises a mixture of ethanol and water and further comprises polyethylene glycol 300 or polyethylene glycol 400, specifically in a concentration of from about 150 mg/mL to about 250 mg/mL, from about 175 mg/mL to about 225 mg/mL, or about 200 mg/mL, and the pH of the medically active liquid is from about 4.0 to about 5.0, from about 4.25 to about 4.75, or about 4.5. In further specific embodiments, the mixture of ethanol and water is an about 70:30 (w/w %) ethanol:water mixture, an about 80:20 (w/w %) ethanol:water mixture, or an ethanol:water mixture in the range from about 70:30 (w/w %) ethanol:water to about 80:20 (w/w %) ethanol:water. In further specific embodiments, the mixture of ethanol and water is an about 70:30 (w/w %) ethanol:water mixture. In yet further specific embodiments, the medically active liquid does not comprise a non-ionic surfactant, such as a polysorbate surfactant, for example, polysorbate 80.

In further specific embodiments, the medically active liquid to be administered according to the present invention may be essentially free of a propellant, such as a hydrofluoroalkane (HFA) propellant.

The inhalable corticosteroid or, more specifically, the medically active liquid to be administered according to the present invention is usually administered in 1 to 4 doses per day, or 2 or 3 doses per day using an inhaler or inhalation device as described in further detail below. Accordingly, in further specific embodiments, one dose of the medically active liquid comprises the selected inhalable corticosteroid, preferably selected from beclomethasone, budesonide, dexamethasone, fluticasone, mometasone (furoate) and/or ciclesonide, especially ciclesonide, or the selected combination of inhalable corticosteroids in an amount selected within the range of from about 25 μg to about 500 μg, specifically from about 40 μg to about 400 μg or even more specifically from about 50 μg to about 350 μg.

In some embodiments, the medically active liquid comprising the inhalable corticosteroid, preferably selected from beclomethasone, budesonide, dexamethasone, fluticasone, mometasone (furoate) and/or ciclesonide, more preferably ciclesonide, is dispensed and/or administered in an amount of at least about 1 μL, 2 μL, 5 μL, 10 μL, or 15 μL, or at least about 20 μL, 25 μL, 30 μL, or 50 μL, or from about 1 μL to about 50 μL or from about 2 μL to about 30 μL, or from about 5 μL to about 25 μL, or from about 10 μL to about 20 μL. In some embodiments, the medically active liquid comprising the inhalable corticosteroid, preferably ciclesonide, is dispensed and/or administered in an amount of about 15 μL.

In further specific embodiments, the medically active liquid according to the present invention may generally comprise the selected inhalable corticosteroid, preferably or the selected combination of inhalable corticosteroids in a concentration selected within the range of from about 0.1 μg/μL to about 100 μg/μL, such as from about 0.5 μg/μL to about 90 μg/μL, or even from about 1 μg/μL to about 80 μg/μL, or from about 2 μg/mL to about 70 μg/mL, especially in cases in which a binary solvent system as described above comprising ethanol and water as the only solvents are used as the liquid vehicle.

In further specific embodiments in which the selected inhalable corticosteroid is ciclesonide, the concentration may be selected within the range of from about 1 μg/μL to about 80 μg/μL, such as from about 5 μg/μL to about 70 μg/μL, or even from about 15 or 20 μg/μL to about 65 μg/μL, or from about 5 μg/μL to about 25 μg/μL, especially in cases in which a binary solvent system as described above comprising ethanol and water as the only solvents are used as the liquid vehicle.

In further specific embodiments in which the selected inhalable corticosteroid is mometasone or fluticasone proprionate or fluticasone furoate, the concentration may be selected within the range of from about 0.5 μg/μL to about 10 μg/μL, such as from about 1 μg/μL to about 7.5 μg/μL, or even from about 1.5 or 2 μg/μL to about 6 μg/μL, especially in cases in which a binary solvent system as described above comprising ethanol and water as the only solvents are used as the liquid vehicle.

In some embodiments, the concentration of the inhalable corticosteroid, preferably ciclesonide, in the medically active liquid is about 1 μg/μL, 2 μg/μL, 3 μg/μL, 4 μg/μL, 5 μg/μL, 6 μg/μL, 7 μg/μL, 8 μg/μL, 9 μg/μL, 10 μg/μL, 11 μg/μL, 12 μg/μL, 13 μg/μL, 14 μg/μL, 15 μg/μL, 16 μg/μL, 17 μg/μL, 18 μg/μL, 19 μg/μL or 20 μg/μL.

In specific embodiments, the selected inhalable corticosteroid or, more specifically the medically active liquid comprising the inhalable corticosteroid and optionally the further pharmaceutically active components or excipients as described above may be administered for prolonged periods of time such as for several weeks or even months, depending on severity and success of the treatment of the subject in need thereof. In further specific embodiments, however, the inhalable corticosteroid of the medically active liquid comprising such corticosteroid is preferably administered for a period of at least 5 days, such as from 5 to about 14 days or to about 10 days.

According to the present invention, the medically active liquid comprising the inhalable corticosteroid is administered to the subject in need of such administration by inhalation of the medically active liquid in nebulized form. Such nebulization and administration by inhalation can be performed using an inhalation device, specifically a hand-held inhalation device. The term “inhalation device” as used herein is to be understood in the broadest sense as referring to a device which is configured and adapted for the generation of an inhalable mist, vapor, or spray, or more specifically, a device that allows and is adapted for the nebulization in inhalative administration, preferably by oral inhalation, of a medically active liquid. Examples of such inhalation devices are known to those of skill in the art and comprise, but are not limited to, e.g., metered dose inhalers (MDI), nebulizers, vibrating mesh inhalers and soft-mist-inhalers (SMI). Exemplary embodiments of suitable inhalers for the administration of the medically active liquid comprising an inhalable corticosteroid, preferably ciclesonide, are described, e.g., in “Inhalation drug delivery devices: technology update” Medical Devices: Evidence and Research 2015:8 131-139; or “Recent advances in in aerosolized drug delivery”, A. Chandel et al., Biomedicine & Pharmacotherapy, Vol. 112, April 2019, 108601 (doi.org/j.biopha.2019.108601), or in “Pharmaceutical Inhalation Aerosol Technology”, Third Edition, A. J. Hickey et al., May 1, 2019, the contents of each of which are herein incorporated by reference in their entireties.

Accordingly, in specific embodiments of the present invention the medically active liquid to be administered to the subject in nebulized form may be generated using an inhalation device, specifically a hand-held inhalation device. Suitable inhalation devices comprise soft-mist inhalers (SMIs). The term “soft-mist-inhaler” as used herein, in specific embodiments, refers to a preferably non-electrified mobile inhalation device for liquid formulations with low velocity nebulization properties. In further specific embodiments, such inhalation device or, more specifically, such soft-mist inhaler comprises at least one impingement-type nozzle as described in further detail below for the nebulization/aerosolization of the medically active liquid.

Soft-mist inhalers as described above have been proven as a very effective means for providing medically active liquids or compositions or pharmaceutically active compounds contained therein into the lung of a patient or subject in need thereof. A soft-mist inhaler as referred to herein typically comprises one or a plurality of impingement-type nozzles. Such an impingement-type nozzle is adapted to emit at least two jets of liquid which are directed such as to collide and break up into small aerosol droplets, thereby generating an aerosol of the medically active liquid in nebulized or aerosolized form. Especially in the case of hand-held nebulizers the nozzle or nozzles usually are firmly affixed to the user-facing side of the housing of the inhalation device in such a way that it is immobile, or non-moveable, relative to the housing or at least relative to the side or part of the housing which faces the user (e.g., patient) when the device is used.

Other suitable inhalation devices are known such as, e.g., the Respimat® inhaler (Boehringer Ingelheim), vibrating membrane nebulizers such as eFlow® (PARI), Vibrating-Mesh® nebulizers (such as Philips InnoSpire Go) and others.

A further exemplary suitable inhalation device is known, e.g., from document EP 0 627 230 B1, the contents of which are incorporated herein by reference in its entirety. Essential components of this exemplary inhalation device are a reservoir in which the medically active liquid that is to be aerosolized is contained; a pumping device for generation of a pressure being sufficiently high for nebulizing; as well as an atomizing device in the form of a nozzle. By means of the pumping device, the liquid is drawn in a discrete amount, i.e., not continuously, from the reservoir, and fed to the nozzle. The pumping device works without propellant and generates pressure mechanically. Accordingly, in specific embodiments a preferred inhalation device to be used in the context of the present invention works without a propellant. In further specific embodiments, the pressure of the medically active liquid to be dispensed is generated mechanically, such as by the force of a spring.

A further exemplary embodiment of a suitable inhalation device is described in document WO 91/14468 A1, the contents of which are herein incorporated by reference in its entirety. In such a device, the pressure in the pumping chamber which is connected to the housing is generated by movement of a moveable hollow piston. The piston is moveably arranged inside the immobile cylinder or pumping chamber. The (upstream arranged) inlet of the hollow piston is fluidically connected to the interior of the reservoir (reservoir pipe section). Its (downstream arranged) tip leads into the pumping chamber. Furthermore, a check valve that inhibits a back flow of liquid into the reservoir is arranged inside the tip of the piston.

A specific embodiment of such a soft mist inhaler which is suitable for the administration of the medically active liquid comprising an inhalable corticosteroid, preferably selected from beclomethasone, budesonide, dexamethasone, fluticasone, mometasone (furoate) and/or ciclesonide, especially ciclesonide, is described, e.g., in international patent application WO 2018/197730 A1, the contents of which are incorporated herein by reference in its entirety. It should be noted, however, that the inhaler device described therein is just one example of a suitable inhaler device to be used according to the present invention and, therefore should not be interpreted as limiting the scope of the invention in any respect.

Accordingly, in specific embodiments, the inhalation device that may be used in the context of the present invention to administer the medically active liquid comprising an inhalable corticosteroid may be a hand-held inhalation device for delivering a nebulized medically active aerosol for inhalation therapy, comprising

(a) a housing having a user-facing side;

(b) an impingement-type nozzle for generating the nebulized aerosol by collision of at least two liquid jets, the nozzle being firmly affixed to the user-facing side of the housing such as to be immobile relative to the housing;

(c) a fluid reservoir arranged within the housing; and

(d) a pumping unit arranged within the housing, the pumping unit having
an upstream end that is fluidically connected to the fluid reservoir;
a downstream end that is fluidically connected to the nozzle;
   wherein the pumping unit is adapted for pumping fluid from the fluid reservoir to the nozzle;

wherein the pumping unit further comprises

(i) a riser pipe having an upstream end, wherein the riser pipe is
adapted to function as a piston in the pumping unit, and
firmly affixed to the user-facing side of the housing such as to be immobile relative to the housing; and

(ii) a hollow cylinder located upstream of the riser pipe, wherein the upstream end of the riser pipe is inserted in the cylinder such that the cylinder is longitudinally movable on the riser pipe; and

(iii) a lockable means for storing potential energy when locked and for releasing the stored energy when unlocked, the means being arranged outside of, and mechanically coupled to, the cylinder such that unlocking the means results in a propulsive longitudinal movement of the cylinder towards the downstream end of the pumping unit.

Such a preferred inhalation device comprises a housing having a user-facing side, an impingement-type nozzle for generating the nebulized aerosol by collision of at least two liquid jets, a fluid reservoir arranged within the housing, and a pumping unit which is also arranged within the housing. In these preferred embodiments, the nozzle may be firmly affixed to the user-facing side of the housing such as to be immobile relative to the housing. The pumping unit may have an upstream end that is fluidically connected to the fluid reservoir and a downstream end that is fluidically connected to the nozzle, whereas in the context of the present invention an “upstream” direction or position means a position or direction from which the medically active liquid is conveyed, and a “downstream” direction or position means a position or direction to which the medically active liquid is conveyed or in other words in the direction of the nozzle. Furthermore, the pumping unit may be adapted for pumping fluid from the fluid reservoir to the nozzle, and it may comprise a riser pipe which is adapted to function as a piston in the pumping unit, a hollow cylinder and a lockable means for storing potential energy. The riser pipe may be firmly affixed to the user-facing side of the housing such as to be immobile relative to the housing. The hollow cylinder may be located upstream of the riser pipe, and the upstream end of the riser pipe may be inserted in the cylinder such that the cylinder is longitudinally movable on the riser pipe. The lockable means typically is capable of storing potential energy when locked and adapted for releasing the stored energy when unlocked. The means may be arranged outside of, and mechanically coupled to, the cylinder in such a way that unlocking the means results in a propulsive longitudinal movement of the cylinder towards the downstream end of the pumping unit.

As used herein, a “hand-held” inhalation device is a mobile inhalation device which can be conveniently held in one hand (preferably by the user but also by another person) and which is suitable for delivering a nebulized medically active aerosol for inhalation therapy. In order to be suitable for inhalation therapy, the device must be able to emit a medically active aerosol, namely the medically active liquid of the present invention in nebulized form, whose particle or, more specifically, droplet size is respirable, i.e., small enough to be taken up by the lungs of a patient or user. Typically, respirable particles have a diameter as measured by laser diffraction of not more than about 10 μm, in particular not more than about 7 μm, or not more than about 5 μm, respectively. In this respect, inhalation devices suitable for the administration of the medically active liquid in nebulized form according to the present invention are also substantially different from devices that emit spray for oral or nasal administration, such as disclosed in US 2004/0068222 A1.

In some embodiments, a Dv50 value of the nebulised medically active aerosol comprising an inhalable corticosteroid, preferably selected from beclomethasone, budesonide, dexamethasone, fluticasone, mometasone (furoate) and ciclesonide, especially ciclesonide, is from about 2.0 μm to about 7.0 μm. In other embodiments, the Dv50 value of the nebulised medically active aerosol comprising an inhalable corticosteroid, preferably selected from beclomethasone, budesonide, dexamethasone, fluticasone, mometasone (furoate) and ciclesonide, especially ciclesonide, is from about 3.0 μm to about 6.0 μm. In other embodiments, the Dv50 value of the nebulised medically active aerosol comprising an inhalable corticosteroid, preferably selected from beclomethasone, budesonide, dexamethasone, fluticasone, mometasone (furoate) and ciclesonide, especially ciclesonide, is from about 4.0 μm to about 6.0 μm. In other embodiments, the Dv50 value of the nebulised medically active aerosol comprising an inhalable corticosteroid, preferably selected from beclomethasone, budesonide, dexamethasone, fluticasone, mometasone (furoate) and ciclesonide, especially ciclesonide, is from about 5.0 μm to about 6.0 μm. In yet other embodiments, the Dv90 value of the nebulised medically active aerosol comprising an inhalable corticosteroid, preferably selected from beclomethasone, budesonide, dexamethasone, fluticasone, mometasone (furoate) and ciclesonide, especially ciclesonide, is from about 5 μm to about 25 μm, from about 10 μm to about 20 μm, or from about 10 μm to about 15 μm. The terms “Dv10, Dv50, and Dv90” refer to the size point in micrometers (μm) below which 10%, 50%, and 90%, respectively, of which the sample volume exists. In other words, the Dv10 is the size point below which there is 10% of the volume of the sample, the Dv50 is the size point below which there is 50% of the volume of the sample, and the Dv90 is the size point below which there is 90% of the volume of the sample.

The inhalation device that may be used according to the present invention is capable of delivering a nebulized aerosol or, more specifically, the medically active liquid comprising an inhalable corticosteroid in nebulized form. As used herein, an aerosol is a system having at least two phases: a continuous phase which is gaseous, and which comprises a dispersed liquid phase in the form of small liquid droplets. Optionally, the liquid phase may itself represent a liquid solution, dispersion, suspension, or emulsion. In specific embodiments, the gaseous phase of the medically active liquid in aerosolized form according to the present invention is air or another physiologically acceptable gas or a mixture thereof, preferably air.

Important for the generation of a nebulized aerosol is a suitable nozzle. According to specific embodiments of the invention, the nozzle of preferred inhalation devices, specifically soft mist inhalers, is of the impingement type. This means that the nozzle is adapted to emit at least two jets of the medically active liquid which are directed such as to collide and break up into small aerosol droplets. The nozzle may be firmly affixed to the user-facing side of the housing of the inhalation device in such a way that it is immobile, or non-moveable, relative to the housing or at least relative to the side or part of the housing which faces the user (e.g., patient) when the device is used.

The fluid reservoir of the specific hand-held inhalation device as described above which is typically arranged within the housing may be adapted to hold or store the medically active liquid from which the nebulized aerosol is generated and delivered by the inhalation device.

The pumping unit of the specific inhalation device which is also arranged within the housing may be adapted to function as a piston pump, also referred to as plunger pump, wherein the riser pipe may function as the piston, or plunger, which is longitudinally moveable within the hollow cylinder. In this embodiment, the inner segment of the hollow cylinder in which the upstream end of the riser pipe moves forms a pumping chamber which has a variable volume, depending on the position of the riser pipe relative to the cylinder.

The hollow cylinder of the preferred inhalation device which provides the pumping chamber is fluidically connected with the fluid reservoir, either directly or indirectly, such as by means of an optional reservoir pipe (or reservoir pipe section). Similarly, the riser pipe, whose reservoir-facing, interior (upstream) end which can be received in the hollow cylinder, is fluidically connected at its downstream or exterior end to the nozzle in a liquid-tight manner, either directly or indirectly.

In this context, the expression “hollow cylinder” as used herein refers to a part or member which is hollow in the sense that it comprises an internal void which has a cylindrical shape, or which has a segment having a cylindrical space. In other words, and as is applicable to other types of piston pumps, it is not required that the external shape of the respective part or member is cylindrical. Moreover, the expression “hollow cylinder” does not exclude an operational state of the respective part or member in which the “hollow” space may be filled with material, e.g., with a liquid to be nebulized.

As used herein, a “longitudinal movement” is a movement along the main axis of the hollow cylinder, and a propulsive movement is a movement of a part in a downstream (or forward) direction.

Importantly, the riser pipe of the pumping unit of the preferred hand-held inhalation device is arranged downstream of the cylinder, and it is firmly affixed to the user-facing side of the housing such as to be immobile relative to the housing or at least to the part of the housing which comprises the user-facing side of the housing. For the avoidance of doubt, the term “firmly fixed” as used herein means either directly or indirectly (i.e., via one or more connecting parts) fixed such as to prevent relative movement between the respective parts. As in the preferred inhalation device as described above the nozzle is also immobile relative to the housing or the respective part of the housing, the riser pipe is also immobile relative to the nozzle, and the pumping action is affected by the longitudinal movement of the hollow cylinder. A propulsive movement of the cylinder, which is arranged in an upstream position relative to the riser pipe, results in a decrease of the volume of the pumping chamber, and a repulsive movement of the cylinder results in an increase of the volume. In other words, in the preferred hand-held inhalation device the riser pipe maintains its position relative to the housing, and the hollow cylinder can alter its position relative to the housing, and in particular, along a longitudinal axis of the same, such as to perform a piston-in-cylinder-type movement of the immobile riser pipe in the moveable cylindrical member.

This arrangement differs from other impingement-type inhalation devices which rely on a pumping unit whose riser pipe is in an upstream position and a cylindrical member in a downstream position wherein the riser pipe is moveable and the cylindrical member is fixed to the housing, as disclosed in US 2012/0090603 A1. For the avoidance of doubt, however, it should be noted that in specific embodiments these inhalation devices may also be used to nebulize the medically active liquid according to the present invention.

A key advantage of the described preferred inhalation device is that the passage between pumping chamber and fluid reservoir can be designed with less restrictions with respect to its dimensions. It is e.g., possible to accommodate a significantly larger inlet valve (also referred to as check valve), which is easier to manufacture since it does not have to be contained within a narrow riser pipe. Instead, the invention allows the use of a check valve whose size is only restricted by the interior size of the housing or the dimensions of the means for storing potential energy. In other words, the diameters of the valve, the riser pipe and—if used—the reservoir pipe do not need to match each other. Furthermore, since no movable piston needs to be connected to the fluid reservoir, the component which provides the fluid connection to the reservoir can be designed independently of the moveable component, i.e., the hollow cylinder, allowing the individual parts to be adapted to suit their respective individual functions. In this respect, the preferred inhalation device as described above provides for higher design flexibility because the moveable hollow cylinder, due to its robust structure and dimensions, provides better opportunities for designing a mechanically stable connection with the reservoir than would a less robust moveable riser pipe. Also, the connection between the hollow cylinder and the fluid reservoir can be designed with a larger diameter, such that higher flow velocities and fluid viscosities become feasible. Further, a support for the reservoir can be integrated into any component that comprises the cylinder. Additionally, any vent for pressure equilibration of the reservoir can be moved away from the reservoir body itself to (e.g.) a connector which forms an interface between reservoir and hollow cylinder, thus facilitating construction and avoiding the necessity to provide an essentially “open” reservoir body.

As already mentioned, the lockable means for storing potential energy of the preferred inhalation device is adapted to store energy in its locked state and to release the stored energy when unlocked. In specific embodiments, the lockable means is mechanically coupled to the hollow cylinder in such a way such that unlocking the means results in a propulsive longitudinal movement of the cylinder towards the downstream end of the pumping unit. During this movement, the internal volume of the cylinder, i.e., the volume of the pumping chamber, decreases. Vice versa, when the means for storing potential energy is in the locked state, the hollow cylinder is in its most upstream position in which the volume of the pumping chamber is largest. The locked state could also be considered a primed state. When the state of the means for storing energy is altered from the unlocked to the locked state, which could be referred to as priming the device, the hollow cylinder performs a repulsive longitudinal movement, i.e., from its most downstream position towards its most upstream position. A pumping cycle of the preferred inhalation device as described above consists of two subsequent and opposing movements of the cylinder starting from its most downstream position to its most upstream (or primed) position and—driven by the means for storing potential energy that now releases its energy—back to its most downstream position.

In specific embodiments, the inhalation device suitable for the generation of the medically active liquid in nebulized form according to the present invention is capable, especially in the case of inhalation device having an impingement-type nozzle is capable of pressurizing the medically active liquid to be nebulized to a pressure of up to 1,000 bar (one thousand bar), such as from about 2 bar to about 500 bar or to about 300 bar or from about 50 bar to about 250 bar.

In specific embodiments of the preferred inhalation device as described above, the pumping unit is a high-pressure pumping unit and adapted to operate, or to expel fluid, at a pressure of at least about 50 bar. In other preferred embodiments, the operating pressure of the pumping unit is at least about 10 bar, or at least about 100 bar, or from about 2 bar to about 1,000 bar, or from about 50 bar to about 250 bar, respectively. As used herein, the “operating” pressure is the pressure at which the pumping unit expels fluid, in particular the medically active liquid comprising an inhalable corticosteroid, from its pumping chamber in a downstream direction, i.e., towards the nozzle. In this context, the expression “adapted to operate” means that the components of the pumping unit are selected with respect to the materials, the dimensions, the quality of the surfaces and the finish are selected such as to enable operation at the specified pressure.

Moreover, such high-pressure pumping unit implies that the means for storing potential energy preferably is capable of storing and releasing a sufficient amount of energy to drive the propulsive longitudinal movement of the cylinder with such a force that the respective pressure is obtained.

For example, in the preferred inhalation device as described herein the means for the storage of potential energy may be designed as tension or pressure spring. Alternatively, besides a metallic or plastic body, also a gaseous medium, or magnetic force utilizing material can be used as means for energy storage. By compressing or tensioning, potential energy may be fed to the means. One end of the means may be supported at or in the housing at a suitable location; thus, this end is essentially immobile. With the other end, it may be connected to the hollow cylinder which provides the pumping chamber; thus, this end is essentially moveable. The means can be locked after being loaded with a sufficient amount of energy, such that the energy can be stored until unlocking takes place. When unlocked, the means can release the potential energy (e.g. spring energy) to the cylinder with the pumping chamber, which is then driven such as to perform a (in this case, longitudinal) movement. Typically, the energy release takes place abruptly, so that a high pressure can build up inside the pumping chamber before a significant amount of liquid is emitted, which results in a pressure decrease. In the preferred inhalation device as described above, during a significant portion of the ejection phase, an equilibrium exists of pressure delivered by the means for the storage of potential energy, and the amount of already emitted liquid. Thus, the amount of liquid remains essentially constant during this phase, which is a significant advantage to devices which use manual force of the user for the emission, such as the devices disclosed in documents US 2005/0039738 A1, US 2009/0216183 A1, US 2004/0068222 A1, or US 2012/0298694 A1, since manual force depends on the individual user or patient and is very likely to vary largely during the ejection phase, resulting in inhomogeneous droplet formation, size, and amount. In contrast to these devices, the means according to the preferred inhalation device as described above in connection with the present invention ensures that the inhalation device delivers highly reproducible results.

The means for storing potential energy may also be provided in the form of a highly pressurized gas container. By suitable arrangement and repeatable intermittent activating (opening) of the same, part of the energy which is stored inside the gas container can be released to the cylinder. This process can be repeated until the remaining energy is insufficient for once again building up a desired pressure in the pumping chamber. After this, the gas container must be refilled or exchanged.

In one of the preferred embodiments, the means for storing potential energy comprised by the inhalation device that may be used in the context of the present invention is a spring having a load of at least 10 N in a deflected state. In a particularly preferred embodiment, the means for storing potential energy is a compression spring made of steel having a load from about 1 N to about 500 N in its deflected state. In other preferred embodiments, the compression spring from steel has a load from about 2 N to about 200 N, or from about 10 N to about 100 N, in its deflected state.

The inhalation device that may be used in connection with the present invention is preferably adapted to deliver the nebulized medically active aerosol (i.e., the medically active liquid comprising an inhalable corticosteroid in nebulized form) in a discontinuous manner, i.e., in the form of discrete units, wherein one unit is delivered per pumping cycle. In this aspect, suitable inhalation devices differ from commonly known nebulizers such as jet nebulizers, ultrasonic nebulizers, vibrating mesh nebulizers, or electrohydrodynamic nebulizers which typically generate and deliver a nebulized aerosol continuously over a period of several seconds up to several minutes, such that the aerosol requires a number of consecutive breathing maneuvers in order to be inhaled by the patient or user. Instead, a preferred inhalation device of the invention is adapted to generate and emit discrete units of aerosol, wherein each of the units corresponds to the amount (i.e., volume) of fluid (i.e., medically active liquid) which is pumped by the pumping unit in one pumping cycle into the nozzle where it is immediately aerosolized and delivered to the user or patient. Vice versa, the amount of liquid pumped by the pumping unit in one pumping cycle determines the amount of the pharmacologically active agent which the patient receives per dosing. It is therefore highly important with respect to achieving the desired therapeutic effect that the pumping unit operates precisely, reliably and reproducibly. The inventors have found that especially the preferred inhalation device as described above incorporating the pumping unit as described above is particularly advantageous in that it does exhibit high precision and reproducibility.

In one preferred embodiment, a single dose of the medication (i.e., of the inhalable corticosteroid, preferably selected from beclomethasone, budesonide, dexamethasone, fluticasone, mometasone (furoate) and ciclesonide, especially ciclesonide, comprised by the nebulized medically active liquid) is contained in one unit, i.e., in the volume that is delivered from the pumping unit to the nozzle for aerosol generation in one single pumping cycle. In this case, the user or patient will prime and actuate the inhalation device only once, and inhale the released aerosol in one breathing maneuver, per dosing (i.e., per dosing event).

In another preferred embodiment, a single dose of the medication consists of two units of the aerosol, and thus requires two pumping cycles. Typically, the user or patient will prime the device, actuate it such as to release and inhale a unit of the aerosol, and then repeat the procedure. Alternatively, three or more aerosol units may constitute a single dosing.

The volume of medically active liquid that is pumped by the pumping unit in one pumping cycle is preferably in the range from about 2 to about 150 μl. In particular, the volume may range from about 0.1 μL to about 1,000 μL (one thousand μL), or from about 1 μL to about 250 μL or from about 1 μL to about 100 μL, or from about 2 μL to about 50 μL, or from about 5 μL to about 25 μL, respectively. These volume ranges are nearly the same as the volume of liquid phase that is contained in one unit of aerosol generated by the inhalation device, perhaps with minor differences due to minute losses of liquid in the device.

In another preferred embodiment of the preferred inhalation device as described above, the pumping unit comprises an inlet valve, also referred to as a check valve or inlet check valve, positioned in the hollow cylinder. According to this embodiment, the interior space of the hollow cylinder, i.e., the pumping chamber, is fluidically connected with the fluid reservoir via the inlet check valve. The inlet valve allows the inflow of liquid into the pumping chamber, but prevents the backflow of liquid towards, or into, the fluid reservoir. The position of the inlet valve may be at or near the upstream end of the cylinder such as to make nearly the entire internal volume of the hollow cylinder available for functioning as the pumping chamber. Alternatively, it may be more centrally located along the (longitudinal) main axis of the hollow cylinder such as to define an upstream segment and a downstream segment of the cylinder, the upstream segment being upstream of the inlet valve and the downstream segment being downstream of the valve. In this case the pumping chamber is located in the downstream segment.

As mentioned, one of the advantageous effects is that an inlet valve having relatively large dimensions may be accommodated in this position, i.e., at the upstream end of the pumping chamber. This is particularly beneficial as it allows for large dimensions of the fluid conduit(s) within the valve, thus enabling high fluid velocities which translate into a rapid filling of the pumping chamber during the priming of the inhalation device. Moreover, the use of liquids having a higher viscosity than ordinary liquid formulations for inhalation, such as highly concentrated solutions of soluble active ingredients, become feasible for inhalation therapy.

According to a further preferred embodiment, the inlet valve is adapted to open only when the pressure difference between the upstream and the downstream side of the valve, i.e., the fluid reservoir side and the pumping chamber side, is above a predefined threshold value, and remains closed as long as the pressure difference is below the threshold value. The term “pressure difference” as used in that context means that, irrespective of the absolute pressure values, only the relative pressure difference between the two sides is relevant for determining whether the valve blocks or opens. If, for example, the pressure on the upstream (reservoir) side is already positive (e.g., 1.01 bar due to thermal expansion), but the pressure on the downstream (pumping chamber) side is ambient pressure (1.0 bar, no activation of the device), the pressure difference (here: 0.01 bar) is below the threshold value (e.g., 20 mbar), which allows the valve to stay closed even when subject to a positive pressure in opening direction. This means that the check valve remains closed until the threshold pressure is met, thus keeping the passage between reservoir and pumping chamber safely shut e.g., when the inhalation device is not in use. Examples for threshold pressure differences are in the range of 1 to 1,000 mbar, and more preferably between about 10 and about 500 mbar, or between about 1 and about 20 mbar.

When actuating the above-described inhalation device, as the means for storing potential energy alters its state from a locked state to an unlocked state, energy is released which effects the cylinder to perform its propulsive longitudinal movement, significant pressure is built up in the pumping chamber. This generates a marked pressure difference (due to a high pressure in the pumping chamber and a substantially lower pressure in the fluid reservoir) which exceeds the threshold value of the pressure difference, so that the check valve opens and allows the pressure chamber to become filled with liquid from the reservoir.

A valve type that may be designed to operate with such a threshold pressure difference is a ball valve pre-loaded with a spring. The spring pushes the ball into its seat, and only if the pressure acting against the spring force exceeds the latter, the ball valve opens. Other valve types which—depending on their construction—may operate with such a threshold pressure difference are duckbill valves or flap valves.

The advantage of such a valve operating with a threshold pressure difference is that the reservoir can be kept closed until active use is being made of the inhalation device, thus reducing unwanted splashing of reservoir liquid during device transport, or evaporation during long-term storage of the device.

In a further preferred embodiment, the inhalation device that may be preferably used in the context of the invention further comprises an outlet valve inside the riser pipe, or at an end of the riser pipe, for avoiding a return flow of liquid or air from the riser pipe into the hollow cylinder. In many cases, the use of such outlet valve will prove to be advantageous. Typically, the downstream end of the riser pipe is located close to the nozzle. The nozzle is in fluidic communication with the outside air. After emitting, in aerosolized form, the amount of liquid which is delivered from the pumping unit through the nozzle, driven by the propulsive longitudinal movement of the cylinder, the pumping chamber must be refilled. For this purpose, it slides back on the riser pipe into its previous upstream position (i.e., performs a repulsive longitudinal movement), so that the interior volume of the pumping chamber increases. Along with this, a negative pressure (sometimes also referred to as “underpressure”) is generated inside the pumping chamber which causes liquid to be sucked into the pumping chamber from the fluid reservoir which is located upstream of the pumping chamber. However, such negative pressure may also propagate downstream through the riser pipe up to the outside of the nozzle and could lead to air being sucked into the device through the nozzle, or nozzle openings, respectively. This problem can be avoided by providing an outlet valve, also referred to as outlet check valve, which opens towards the nozzle openings and blocks in the opposite direction.

Optionally, the outlet valve is of a type that blocks below (and opens above) a threshold pressure difference as described in the context of the inlet valve above. If a ball valve with a spring is used, the spring force must be directed against the pumping chamber such that when the difference between the interior pressure of the pumping chamber and the ambient pressure exceeds the threshold pressure difference value, the outlet valve opens. The advantages of such a valve correspond to the respective aforementioned advantages.

As mentioned, the outlet valve may be positioned within the riser pipe. Alternatively, the inhalation device may comprise an outlet valve which is not integrated within the riser pipe but positioned at or near one of the ends of the riser pipe, in particular at or near its downstream end, e.g., in a separate connector between the riser pipe and the nozzle. This embodiment may be advantageous in certain cases, e.g., if there is a need for a riser pipe with a particularly small diameter which makes the integration of a valve difficult. By accommodating the outlet valve downstream of the riser pipe, a valve with a relatively large diameter may be used, thus simplifying the requirements for the valve design.

In a further alternative embodiment, the outlet valve is absent. This embodiment may be feasible as the fluid channels of an impingement-type nozzle may have relatively small cross sections, resulting in only minor or very slow back flow at the given pressure conditions during the priming of the device. If the amount of backflow is considered acceptable in view of a particular product application, the inhaler design may be simplified by avoiding the outlet valve.

In any case, whether the inhalation device is designed with or without an outlet valve, all other options and preferences described with respect to other device features are applicable to both of these alternative embodiments.

In a further preferred embodiment, the inhalation device that may be used in the context of the present invention comprises a fluid reservoir which is firmly attached to the hollow cylinder such as to be moveable together with the hollow cylinder inside the housing. This means that in each ejection phase of the pumping cycle, the fluid reservoir moves together with the hollow cylinder from an initial (“upstream”) position, in which the pumping chamber has its maximum interior volume, towards an end (“downstream”) position, in which the volume of the pumping chamber is minimal; and during the subsequent “priming” step, the fluid reservoir returns together with the hollow cylinder to their initial (“upstream”) position.

As used herein, the expression “firmly attached” includes both permanent and non-permanent (i.e., releasable) forms of attachment. Moreover, it includes direct and indirect (i.e., via one or more connecting parts) types of attachment. At the same time, as mentioned above, “firmly attached” means that the respective parts are fixed to each other in such a way as to substantially prevent their movement relative to each other. In other words, two parts that are firmly attached to each other may only be movable together, and with respect to each other, they are non-movable or immobile.

One of the advantages of this embodiment wherein the fluid reservoir is firmly attached to the hollow cylinder is that it provides the smallest possible dead volume between the reservoir and the pumping chamber.

According to an alternative embodiment, the fluid reservoir may be fluidically connected to the hollow cylinder by means of a flexible tubular element, and firmly attached to the housing. According to this embodiment, the reservoir is not firmly attached to the hollow cylinder and does not move along with it when the cylinder performs its longitudinal movements. Instead, it is firmly, but optionally detachably, directly or indirectly, attached to the housing or to a part of the housing. One advantage of this embodiment is that the energy which is abruptly released upon unlocking the means for storing potential energy solely acts on the hollow cylinder and not on the fluid reservoir. This may be particularly advantageous in cases in which the fluid reservoir in its initial (fully filled state) at the beginning of its usage has a relatively large mass which decreases overuse. A higher acceleration of the hollow cylinder would translate into a higher pressure in the pumping chamber.

For the avoidance of doubt, all other options and preferences described herein-above and below with respect to other device features are applicable to both of these alternatives, i.e., regardless of whether the fluid reservoir is firmly attached to the hollow cylinder or not.

In one embodiment, the fluid reservoir may be designed to be collapsible, such as by means of a flexible or elastic wall. The effect of such design is that upon repeated use of the device which involves progressive emptying of the reservoir, the flexible or elastic wall buckles or folds such as to reduce the internal volume of the reservoir, so that the negative pressure which is necessary for extraction of a certain amount of liquid is not required to increase substantially over the period of use. In particular, the reservoir may be designed as a collapsible bag. The advantage of a collapsible bag is that the pressure inside the reservoir is almost independent of the filling level, and the influence of thermal expansion is almost negligible. Also, the construction of such a reservoir type is rather simple and already well established.

A similar effect can be achieved with a rigid container which has a moveable bottom (or wall) by means of which the interior volume of the reservoir can also be successively reduced.

Soft-mist inhalers such the specific soft-mist inhaler as described in detail above allow for the administration of discrete doses of the medically active liquid comprising in inhalable corticosteroid, specifically selected from beclomethasone, budesonide, dexamethasone, fluticasone, mometasone (furoate) and ciclesonide, especially ciclesonide, in short periods of time as the generation of the aerosol of the medically active liquid to be administered by inhalation is usually completed within a period (also referred to herein as “spray duration” or “event duration”) of up to 3 sec (seconds), typically within a period selected within the range of from about 0.5 to about 3 sec, or from about 0.5 or from about 1 to about 2 sec.

In a further aspect, the present invention provides for a method for the treatment of a respiratory disease, disorder, or condition resulting from a viral infection in a subject, the method comprising the step of administering to said subject a medically active liquid in nebulized form by inhalation, wherein the medically active liquid comprises an inhalable corticosteroid (ICS), preferably selected from beclomethasone, budesonide, dexamethasone, fluticasone, mometasone (furoate) and ciclesonide, especially ciclesonide, and wherein the medically active liquid is administered in nebulized form using an inhalation device.

In a further aspect, the invention relates to a use of a medically active liquid comprising an inhalable corticosteroid (ICS), preferably selected from beclomethasone, budesonide, dexamethasone, fluticasone, mometasone (furoate) and ciclesonide, especially ciclesonide, for the treatment of a respiratory disease, disorder or condition resulting from a viral infection in a subject, wherein the medically active liquid is used by inhalation of the medically active liquid in nebulized form, wherein the medically active liquid in nebulized form is generated by nebulization using an inhalation device.

In yet a further aspect, the invention relates to a use of an inhalable corticosteroid (ICS), preferably selected from beclomethasone, budesonide, dexamethasone, fluticasone, mometasone (furoate) and ciclesonide, especially ciclesonide, in the manufacture of a medically active liquid for the treatment of a respiratory disease, disorder or condition resulting from a viral infection in a subject, wherein the medically active liquid is administered to the subject in nebulized form by inhalation, and wherein the medically active liquid in nebulized form is generated using an inhalation device.

In yet a further aspect, the present invention provides for a kit, the kit comprising
a medically active liquid comprising an inhalable corticosteroid (ICS) for the treatment of a respiratory disease, disorder or condition resulting from a viral infection in a subject, preferably selected from beclomethasone, budesonide, dexamethasone, fluticasone, mometasone (furoate) and ciclesonide, especially ciclesonide, wherein the medically active liquid is administered to the subject in nebulized form by inhalation; and
an inhalation device, preferably a hand-held inhalation device, such as a soft-mist inhaler.

In a further aspect, the present invention provides for the use of a medically active liquid comprising an inhalable corticosteroid (ICS), preferably selected from beclomethasone, budesonide, dexamethasone, fluticasone, mometasone (furoate) and ciclesonide, especially ciclesonide, for the treatment of a respiratory disease, disorder or condition resulting from a viral infection in a subject, preferably ciclesonide, in the manufacture of a kit comprising a medically active liquid comprising an inhalable corticosteroid (ICS) for the treatment of a respiratory disease, disorder or condition resulting from a viral infection in a subject and an inhalation device, preferably a hand-held inhalation device, such as a soft-mist inhaler.

### DETAILED DESCRIPTION OF THE DRAWINGS

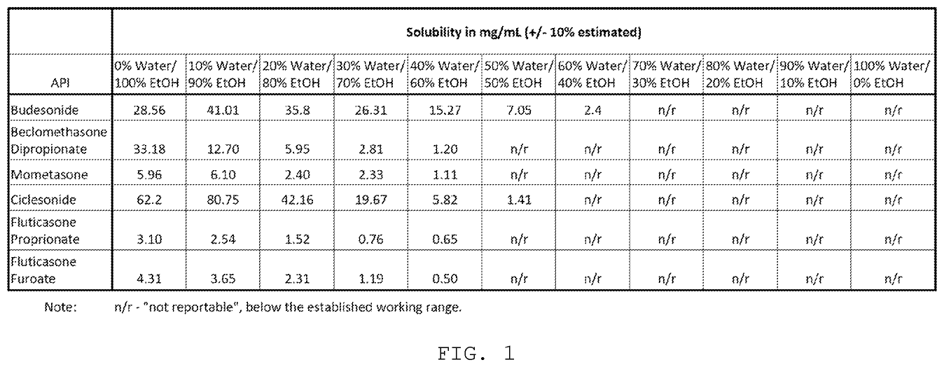

FIG. 1 shows the solubilities of various inhalable corticosteroids in various ethanolic solvent systems as described in Example 1 suitable for the inhalative administration by a soft-mist inhaler.

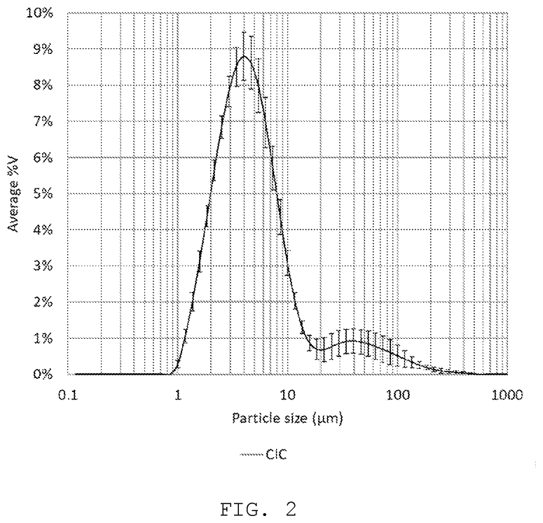

FIG. 2 shows the average particle (drop) size distribution and entire spray duration for ciclesonide in 70:30 ethanol-water via soft-mist inhaler nebulization, measured by laser diffraction as described in Example 2.

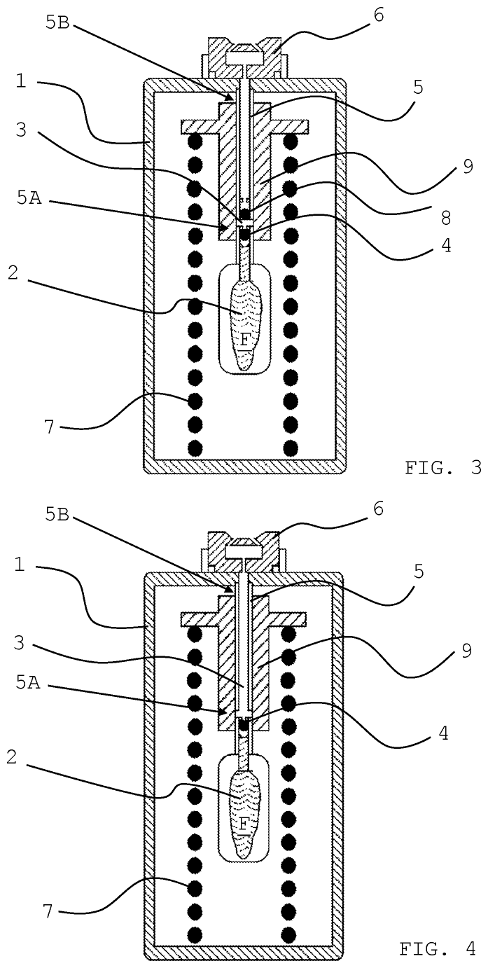

In FIG. 3, one of the preferred embodiments of an inhalation device useful for the nebulization of the medically active liquid according to the present invention is depicted schematically and not-to-scale. FIG. 3 shows the situation prior to first use.

The inhalation device comprises a housing (1), which is preferably shaped and dimensioned such that it can be held with one hand and can be operated by one finger, e.g., a thumb or index finger (not shown). A fluid reservoir (2) for the storage of the medically active liquid (F) to be administered according to the present invention is located inside the housing (1). The depicted reservoir (2) is designed to be collapsible so that in the course of the emptying of the reservoir by the repeated use of the device, the soft or elastic walls deform such that the negative pressure required for withdrawing liquid from the reservoir remains substantially constant over time. A similar effect could be achieved with a rigid container that has a movable bottom by means of which the interior volume of the reservoir can also be successively reduced (not shown).

Furthermore, the shown inhalation device comprises a pumping unit with a hollow cylinder (9) within the housing (1) which forms a pumping chamber (3) for the generation of the desired pressure which is necessary for emitting liquid (F) (i.e., the medically active liquid) and nebulising the same. The pumping unit may also comprise further components not depicted in the drawing, such as a push button, locking device, etc.

As a means for the storage of potential energy (7), a spring is provided which is coupled with one end (upwards directed, or downstream) to the cylinder (9) and which is supported at the housing (1) (lower part of the figure).

The shown inhalation device further comprises a riser pipe (5) with at least one reservoir-facing, or upstream, interior end (5A) which can be received in said cylinder (9). In other words, riser pipe (5) can be at least partially pushed into hollow cylinder (9), resulting in a decrease of the interior volume of pumping chamber (3). The term “interior volume” describes the volume of the space which extends from the reservoir-facing inlet of the cylinder (9) to the place where the interior end (5A) of the riser pipe (5) is located. In the depicted situation, riser pipe (5) is almost entirely contained in the cylinder (9). As a result, the interior volume of the pumping chamber (3), situated between inlet valve (4) and the interior end (5A) of riser pipe (5), is at a minimum.

Preferably, the section (or segment) of the hollow cylinder (9) which serves as, or accommodates, the pumping chamber (3) and which receives the riser pipe (5) exhibits a circular inner cross-section whose diameter relatively closely (e.g., except for a small gap) matches the diameter of the circular outer cross-section of the corresponding segment of the riser pipe (5). Of course, other (e.g., non-circular) cross section shapes are possible as well.

According to the depicted embodiment, inlet valve (4) is arranged between reservoir (2) and inlet of the pumping chamber (3) formed by the cylinder (9).

Furthermore, the inhalation device comprises a nozzle (6) which is connected liquid-tight to the exterior (or downstream) end (5B) of the riser pipe (5). Nozzle (6) is an impingement-type nozzle for generating the nebulised aerosol by collision of at least two liquid jets. Preferably, the cross sections of the liquid-containing channels are relatively small, typically in the region of microns.

Also depicted is an optional outlet valve (8) inside the riser pipe (5) for avoiding a backflow of liquid or air into the exterior end (5B) of the same from the outside. Outlet valve (8) is arranged in the interior end (5A) of riser pipe (5). Liquid (F) can pass outlet valve (8) in direction of nozzle (6), but outlet valve (8) blocks any undesired backflow in the opposite direction.

As can be seen in FIG. 3, riser pipe (5) is designed immobile with respect to the housing (1), and firmly attached to housing (1), indicated by the connection in the region of exterior end (5B) with housing (1). Riser pipe (5) is also firmly attached to nozzle (6), which, in turn, is attached to housing (1) as well. In contrast, the hollow cylinder (9) providing the pumping chamber (3) is designed to be moveable with respect to housing (1) and nozzle (6). The benefits of this design have been explained; reference is made to the respective sections of the description above.

Referring to FIG. 4, a device similar to the one of FIG. 3 is depicted. However, the embodiment shown in FIG. 3 lacks the (optional) outlet valve (8). All other components are present, and also the function is comparable. In this embodiment, pumping chamber (3) extends from downstream of the valve (4) up to nozzle (6), which is the location where the fluidic resistance increases significantly. In an alternative embodiment having a particularly small inner diameter of riser pipe (5), pumping chamber (3) extends only from downstream of the valve (4) up to upstream interior end (5A) of riser pipe (5).

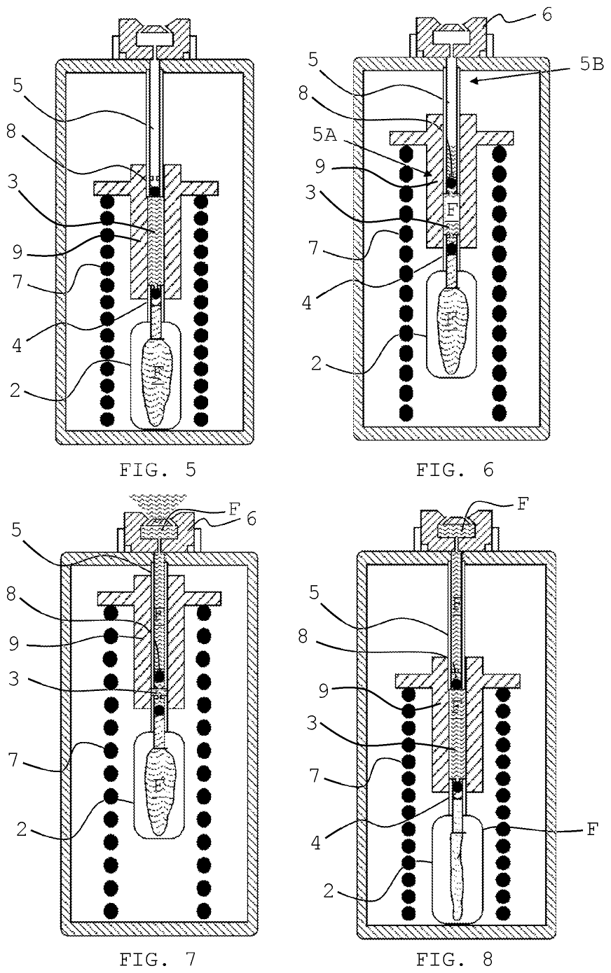

FIG. 5 shows the embodiment of FIG. 3 with a filled pumping chamber. The hollow cylinder (9) has been moved to its most upstream position, thereby loading the means for the storage of potential energy (7). Outlet valve (8) is closed due to negative pressure inside pumping chamber (3), and the inlet valve (4) is open towards the fluid reservoir (2). Increasingly collapsing walls of reservoir (2) allow the internal pressure in the reservoir (2) to remain nearly constant, while the pressure inside the pumping chamber (3) drops because of the propulsive longitudinal motion of the hollow cylinder (9), thus increasing the volume of pumping chamber (3). As a result, the pumping chamber (3) has been filled with the medically active liquid (F) from the reservoir (2).

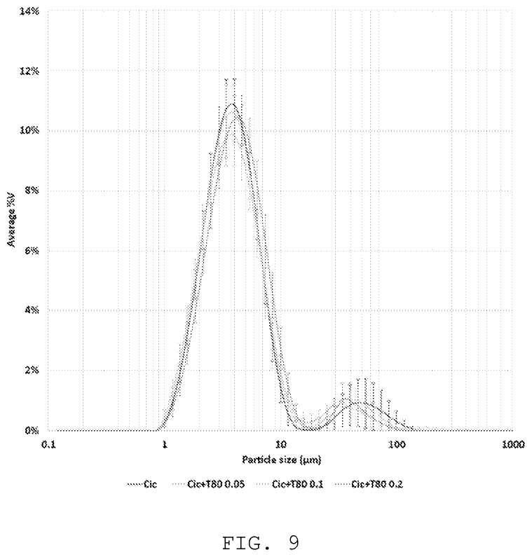

In FIG. 6, the situation after the first actuation of the inhalation device of FIG. 3 is shown. The means for the storage of potential energy (7) has been released from the loaded position as shown in FIG. 5. It pushes the cylinder (9) in a downstream direction such as to slide over the riser pipe (5). The interior end (5A) of the riser pipe (5) has come closer to the inlet check valve (4) which is now closed. As a result, the pressure inside the pumping chamber (3) rises and keeps the inlet valve (4) closed but opens outlet valve (8). Liquid (F) flows from the riser pipe (5) through its exterior end (5B) towards nozzle (6).

FIG. 7 shows the inhalation device of FIG. 3 in the situation at the end of the aerosol emission phase. The means for the storage of potential energy (7) is in its most relaxed end position (spring fully extended). Also, the hollow cylinder (9) has been pushed almost entirely onto riser pipe (5) such that the interior volume of pumping chamber (3) has reached its minimum. Most of the liquid (F) previously contained in the pumping chamber (3) has passed outlet valve (8) into the main segment of the riser pipe (5). Some liquid (F) has been pushed towards, and though, nozzle (6), where nebulisation takes place, such that a nebulised aerosol is emitted towards the user or patient.

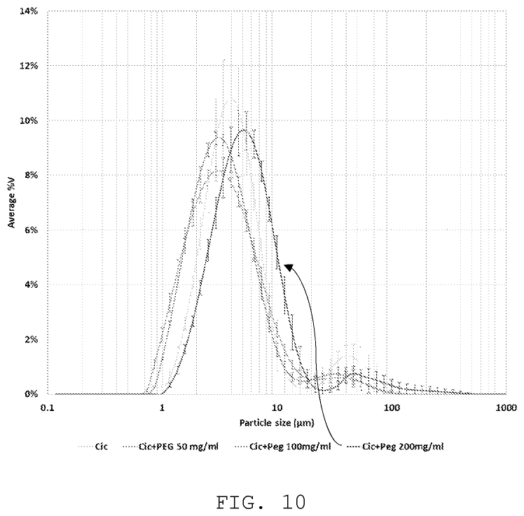

In FIG. 8, the inhalation device of FIG. 3 in the situation after re-filling the pumping chamber is depicted. The hollow cylinder (9) has been moved (repulsively) in an upstream direction, thus increasing the volume of the pumping chamber (3) provided by the cylinder (9). The means for the storage of potential energy (7) has been loaded (spring compressed). During movement of cylinder (9) away from the nozzle (6), a negative pressure has been generated in the pumping chamber (3), closing outlet valve (8) and opening the inlet check valve 4. As a result, further liquid (F) is drawn from reservoir (2) into the pumping chamber (3). The inhalation device's pumping chamber (3) is filled again and ready for the next ejection of liquid (F) by releasing the spring.

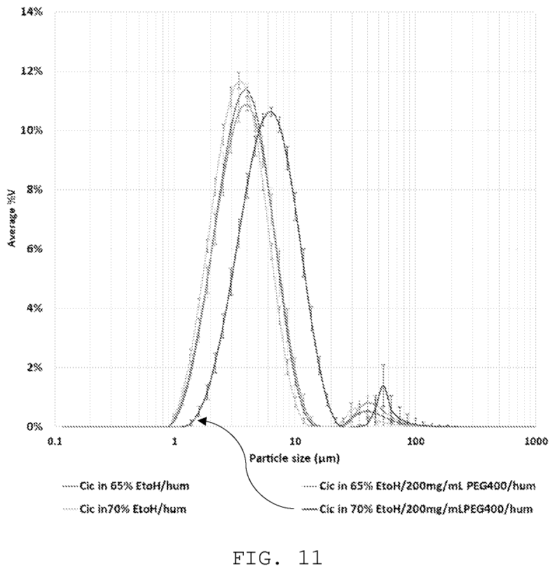

FIG. 9 shows the average particle (drop) size distribution, entire spray duration, for ciclesonide in 70:30 ethanol-water (w/w %) in the presence of Tween 80 via soft-mist inhaler nebulization as measured by laser diffraction as described in Example 5. The presence of Tween 80 did not have a significant impact on the particle (droplet) size. The largest particle size was achieved at the highest Tween 80 concentration (0.200 mg/mL), but the Dv50 value was slightly under 4 μm (3.95).

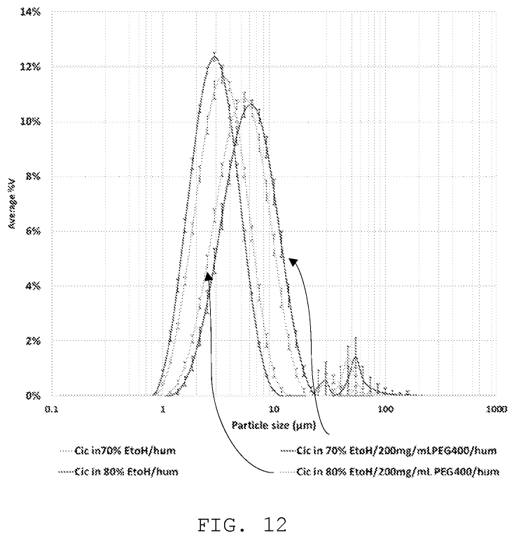

FIG. 10 shows the average particle (drop) size distribution, entire spray duration, for ciclesonide in 70:30 ethanol-water (w/w %) in the presence of PEG300 via soft-mist inhaler nebulization as measured by laser diffraction as described in Example 6. PEG300 had a more significant impact on particle size than Tween 80. A Dv50 value of 4.83 μm was observed at 200 mg/mL PEG300.

FIG. 11 shows the average particle (drop) size distribution, entire spray duration, for ciclesonide in 65:35 and 70:30 ethanol-water (w/w %) mixtures and 65:35 and 70:30 ethanol-water (w/w %) mixtures in the presence of PEG400 via soft-mist inhaler nebulization as measured by laser diffraction as described in Example 7. A shift to higher particle sizes was expected for solutions with higher water concentrations or addition of PEG400 as these reagents would be expected to increase the surface tension of the solutions. This trend was observed when comparing 65% to 70% ethanol:water (w/w %) solutions. However, the addition of PEG to the ethanol:water solutions produces unexpected results. One would expect the 65% ethanol:water solution in the presence of PEG400 to have higher particle sizes than the 70% ethanol:water solution, but the opposite was observed with the 70% ethanol:water solution in the presence of PEG400 producing droplets having a Dv50 value of 5.67 μm compared to 3.57 μm for the 65% ethanol:water solution in the presence of PEG 400.

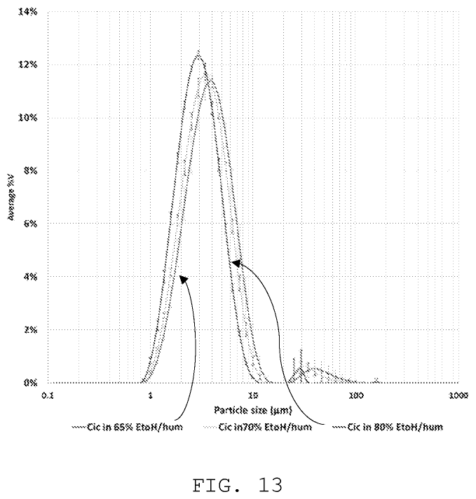

FIG. 12 shows the average particle (drop) size distribution, entire spray duration, for ciclesonide in 70:30 and 80:20 ethanol-water (w/w %) mixtures and 70:30 and 80:20 ethanol-water (w/w %) mixtures in the presence of PEG400 via soft-mist inhaler nebulization as measured by laser diffraction as described in Example 8. Here, expected trends are observed. The 80% ethanol solution produces smaller average particle sizes than the 70% ethanol solution. The 80% ethanol solution in the presence of PEG400 also produces smaller particle sizes than the 70% ethanol solution in the presence of PEG400.

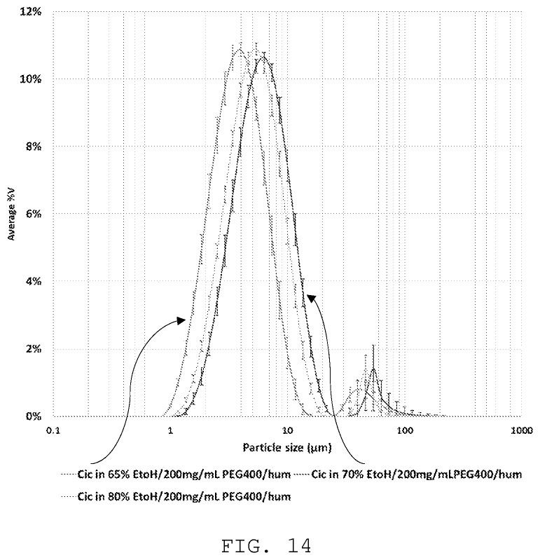

FIG. 13 shows the average particle (drop) size distribution, entire spray duration, for ciclesonide in 65:35, 70:30, and 80:20 ethanol-water (w/w %) mixtures as described in Example 9. As expected, higher average particle sizes are observed at higher (or increasing) water concentrations.

FIG. 14 shows the average particle (drop) size distribution, entire spray duration, for ciclesonide in 65:35, 70:30, and 80:20 ethanol-water (w/w %) mixtures in the presence of PEG400. Unexpected results similar to those discussed in Example 7/FIG. 11 are shown. The presence of PEG400 in the 65%, 70%, and 80% ethanol-water solutions results in a maximum particle size for the 70:30 ethanol:water (w/w %) solution instead of the expected 65:35 ethanol:water (w/w %) solution.

### LIST OF REFERENCES

1 Housing
2 Fluid reservoir, reservoir
3 Pumping chamber
4 Inlet valve
5 Riser pipe
5A Interior end
5B Exterior end
6 Nozzle
7 Means for storing potential energy, means
8 Outlet valve
9 Hollow cylinder, cylinder
F Liquid, fluid, medically active liquid

The following examples serve to illustrate the invention, however, should not be understood as restricting the scope of the invention in any respect:

### EXAMPLES

### Example 1: Solutions Comprising Inhalable Corticosteroids

FIG. 1 shows the solubilities of various inhalable corticosteroids in various ethanolic solvent systems suitable for the inhalative administration by a soft-mist inhaler.

The solutions were prepared by dissolution of the respective inhalable corticosteroids in the various solvent systems as described in FIG. 1 until a saturated solution was obtained. The obtained saturated solutions have been investigated for long-term stability and have proven to be stable over several months. Furthermore, the solutions prepared have been investigated for their suitability for administration using a soft mist inhaler. It was shown that the solutions were suitable for administration in nebulized form using a soft-mist inhaler with a working pressure of at least 200 bar and a spray duration between 1 and 2 sec (seconds). A repeated sprayability test with sufficient fine particle distribution as described in Example 2 below showed no clogging or blocking events of the device.

### Example 2: Spray Tests Using a Solution of Ciclesonide (CIC) in Ethanol:Water (70:30) (w/w) Via Nebulization by a Soft-Mist Inhaler

Table 1 shows the fractions of particle (droplet) sizes when the above-described solution was nebulized with a soft-mist inhaler as described in Example 1. The term “Event duration” as used in Table 1 means the duration of the entire nebulization process in seconds (“Stdev” means standard deviation). The terms “Dv10, Dv50, and Dv90” refer to the size point below which there is 10%, 50%, and 90% of the volume of the sample, respectively. In other words, the Dv10 is the size point below which there is 10% of the volume of the sample, the Dv50 is the size point below which there is 50% of the volume of the sample, and the Dv90 is the size point below which there is 90% of the volume of the sample.

TABLE 1

CIC 70:30
EtOH:H₂O

| | Mean | |
|---|---|---|
| Formulation | (n = 6) | Stdev |
| Event duration/s | 1.91 | 0.07 |
| Dv10/μm | 1.80 | 0.06 |
| Dv50/μm | 4.10 | 0.16 |
| Dv90/μm | 20.47 | 9.01 |

The resulting particle (droplet) size distribution as measured by laser diffraction is summarized in FIG. 2. The graph shows that the maximum of the particle (droplet) size distribution is below 5 μm which allows for good inhalability of the nebulized medically active liquid into the lungs of a subject.

### Example 3: Spray Tests Using Solutions of Further Inhalable Corticosteroids

The solutions of budesonide, beclomethasone diproprionate, mometasone, fluticasone proprionate and fluticasone furoate as summarized in FIG. 1 as well as of dexamethasone are nebulized with a soft-mist inhaler as described in Example 1. A particle size distribution according to the one as described in Example 2 above is expected.

### Example 4: Stability of Ciclesonide Formulations

Formulations of ciclesonide were prepared at various pH levels in either a 100% ethanol solution or a 70:30 (w/w %) ethanol:water mixture. Impurity levels were monitored by HPLC for a period of three months. The results are summarized in Table 2.

TABLE 2

| | | End-total (known) |
|---|---|---|
| Formulation | Composition | impurities (A + B + C) |
| 2.D | Cic + 100% EtOH | 0.899% (3M) |
| 2.E | Cic + 100% EtOH, pH 4.5 | 3.077% (stopped at 2M) |
| 2.F | Cic + 70% EtOH, pH 4.5 | 0.299% (3M) |
| 2.H | Cic + 100% EtOH, pH 2.7 | 7.804% (stopped at 2M) |

Formulations 2.D (100% ethanol) and 2.F (70:30 ethanol:water w/w %) resulted in the lowest end-total (known) impurities.

### Example 5: Spray Tests Using a Solution of Ciclesonide (CIC) in Ethanol:Water (70:30) (w/w) and Tween80 Via Nebulization by a Soft-Mist Inhaler

Although particles below 5 μm allows for good inhalability of the nebulized medically active liquid into the lungs of a subject, smaller particle sizes result in more of the formulation being exhaled instead of staying in the lungs. Therefore, a shift to larger particle sizes may also be desirable. Additives that were hypothesized to shift average particle size distributions to larger values included water, surfactants, and polyethers such as polyethylene glycol.

To investigate the impact of additives to particle size, solutions of ciclesonide in ethanol:water (70:30) (w/w) and Tween80 were prepared by dissolving ciclesonide and Tween80 in the ethanol:water mixture at three concentration levels of Tween80 with a pH adjustment of 4.5. The pH of 4.5 was optimized for ciclesonide stability in solution as outlined in Example 4 above. The compositions of the prepared formulations are shown in Table 3.

TABLE 3

| | Ethanol | Water | | Ciclesonide | Tween80 |
|---|---|---|---|---|---|
| Actual | (%) | (%) | pH | (mg/mL) | (mg/mL) |
| Solution0 | 70 | 30 | 4.50 | 6.22 | 0.00 |
| Solution1 | 70 | 30 | 4.50 | 6.22 | 0.06 |
| Solution2 | 70 | 30 | 4.50 | 6.22 | 0.11 |
| Solution3 | 70 | 30 | 4.50 | 6.22 | 0.22 |

TABLE 4

| | | | Cic + T80 | | Cic + T80 | | Cic + T80 | |
|---|---|---|---|---|---|---|---|---|
| | Cic | | 0.05 | | 0.1 | | 0.2 | |
| | Mean | | Mean | | Mean | | Mean | |
| Param- | (n = | | (n = | | (n = | | (n = | |
| eters | 6) | Stdev | 6) | Stdev | 6) | Stdev | 6) | Stdev |
| Event | 1.83 | 0.02 | 1.88 | 0.07 | 1.77 | 0.19 | 1.81 | 0.20 |
| dura- | | | | | | | | |
| tion/s | | | | | | | | |
| Dv10/μm | 1.79 | 0.06 | 1.70 | 0.08 | 1.76 | 0.10 | 1.90 | 0.09 |
| Dv50/μm | 3.59 | 0.21 | 3.60 | 0.12 | 3.56 | 0.20 | 3.95 | 0.12 |
| Dv90/μm | 16.89 | 15.07 | 11.76 | 6.51 | 8.08 | 1.14 | 10.34 | 4.55 |

The resulting particle (droplet) size distribution as measured by laser diffraction and reported in Table 4 is summarized in FIG. 9. The graph shows that the presence of Tween 80 does not have a great impact on the droplet size until the maximum concentration (0.2 mg/mL) is reached, but even then, the Dv50 value is lower than 4 μm.

### Example 6: Spray Tests Using a Solution of Ciclesonide (CIC) in Ethanol:Water (70:30) (w/w %) and Polyethylene Glycol 300 (PEG300) Via Nebulization by a Soft-Mist Inhaler

To investigate the impact of additives to particle size, solutions of ciclesonide in ethanol:water (70:30) (w/w) and PEG300 were prepared by dissolving ciclesonide in the ethanol:water:PEG300 mixture at three concentration levels of PEG300 with a pH adjustment of 4.5. The composition of the prepared formulations is shown in Table 5.

TABLE 5

| | Ethanol | Water | | Ciclesonide | PEG300 |
|---|---|---|---|---|---|
| Actual | (%) | (%) | pH | (mg/mL) | (mg/mL) |
| Solution4 | 70 | 30 | 4.50 | 6.20 | 0.00 |
| Solution5 | 70 | 30 | 4.50 | 6.20 | 50.10 |
| Solution6 | 70 | 30 | 4.50 | 6.20 | 100.28 |
| Solution7 | 70 | 30 | 4.50 | 6.20 | 201.47 |

Table 6 shows the fractions of particle (droplet) sizes when solutions 4-7 of Table 5 were nebulized with a soft-mist inhaler.

TABLE 6

| | Cic + PEG300 | | Cic + PEG300 | | Cic + PEG300 | |
|---|---|---|---|---|---|---|
| | 50 mg/mL | | 100 mg/mL | | 200 mg/mL | |
| | Mean | | Mean | | Mean | |
| Parameters | (n = 6) | Stdev | (n = 6) | Stdev | (n = 6) | Stdev |
| Event | 2.17 | 0.05 | 2.71 | 0.24 | 2.99 | 0.01 |
| duration/s | | | | | | |
| Dv10/μm | 1.31 | 0.04 | 1.44 | 0.01 | 2.20 | 0.05 |
| Dv50/μm | 3.11 | 0.16 | 3.08 | 0.07 | 4.83 | 0.23 |
| Dv90/μm | 9.13 | 0.76 | 8.56 | 1.70 | 11.93 | 1.91 |

The resulting particle (droplet) size distribution as measured by laser diffraction and reported in Table 6 is summarized in FIG. 10. The graph shows that the presence of PEG300 at the highest concentration (200 mg/mL) has an impact on the droplet size distribution and the Dv50 reaches 4.83 μm. The event duration when adding PEG300 increases due to the higher viscosity of the solution relative to a solution without PEG300.

### Example 7: Spray Tests Using a Solution of Ciclesonide (CIC) in 60%, 65%, and 70% Ethanol:Water Concentrations and Polyethylene Glycol 400 (PEG400) Via Nebulization by a Soft-Mist Inhaler

To investigate the impact of additives to particle size, solutions of ciclesonide in various ethanol:water concentrations and PEG400 were prepared by dissolving ciclesonide in the ethanol:water:PEG400 mixture at one concentration level of PEG400 with a pH adjustment of 4.5. The compositions of the prepared formulations are shown in Table 7. The solution of 60% ethanol was discarded as ciclesonide precipitated from the solution upon cooling in a refrigerator overnight.

TABLE 7

| | | | | Cicles- | Target | Actual |
|---|---|---|---|---|---|---|
| | Ethanol | Water | | onide | PEG400 | PEG400 |
| Actual | (%) | (%) | pH | (mg/mL) | (mg/mL) | (mg/mL) |
| Solution8 | 60 | 40 | 4.52 | 6.21 | 0 | 0.0 |
| Solution9 | 60 | 40 | 4.52 | 6.21 | 200 | 202.0 |
| Solution10 | 65 | 35 | 4.52 | 6.20 | 0 | 0.0 |
| Solution11 | 65 | 35 | 4.52 | 6.20 | 200 | 201.9 |
| Solution12 | 70 | 30 | 4.50 | 6.20 | 0 | 0.0 |
| Solution13 | 70 | 30 | 4.50 | 6.20 | 200 | 201.3 |

Table 8 shows the fractions of particle (droplet) sizes when solutions 10-13 of Table 7 were nebulized with a soft-mist inhaler. All measurements were conducted using humidified air.

TABLE 8

| | Cic 65% | | Cic 65% | | Cic 70% | | Cic 70% | |
|---|---|---|---|---|---|---|---|---|
| | EtOH | | EtOH/PEG400 | | EtOH | | EtOH/PEG400 | |
| | Mean | | Mean | | Mean | | Mean | |
| Param- | (n = | | (n = | | (n = | | (n = | |
| eters | 6) | Stdev | 6) | Stdev | 6) | Stdev | 6) | Stdev |
| Event | 2.03 | 0.02 | 2.74 | 0.21 | 1.89 | 0.12 | 2.98 | 0.01 |
| duration/s | | | | | | | | |
| Dv10/μm | 1.80 | 0.06 | 1.77 | 0.05 | 1.66 | 0.02 | 2.75 | 0.13 |
| Dv50/μm | 3.53 | 0.18 | 3.57 | 0.16 | 3.19 | 0.06 | 5.67 | 0.25 |
| Dv90/μm | 6.95 | 0.52 | 7.34 | 0.46 | 6.18 | 0.26 | 11.87 | 0.96 |

The resulting particle (droplet) size distribution as measured by laser diffraction and reported in Table 8 is summarized in FIG. 11. One would expect higher water concentration and addition of PEG400 would increase particle size because the surface tension of the solution is increased relative to 100% ethanol solutions. This trend is observed when comparing the ciclesonide 65% ethanol:water solution (higher particle sizes) with the 70% ethanol:water solution (lower particle sizes). However, unexpectedly, the addition of PEG to the ethanol:water solutions does not result in the same trend. Instead, the higher ethanol concentration (70% w/w) in combination with the PEG400 gives larger average particle sizes than the 65% ethanol:water/PEG400 formulation, when one would expect the reverse.

### Example 8: Spray Tests Using a Solution of Ciclesonide (CIC) in 70% and 80% Ethanol:Water Solutions and Polyethylene Glycol 400 (PEG400) Via Nebulization by a Soft-Mist Inhaler

To further probe the unexpected results, a higher ethanol concentration of 80% w/w was investigated at the 200 mg/mL ciclesonide concentration and pH of 4.5. Table 9 shows the fractions of particle (droplet) sizes when the 70 or 80% ethanol:water/PEG400 solutions were nebulized with a soft-mist inhaler. All measurements were conducted using humidified air.

TABLE 9

| | Cic 70% | | Cic 70% | | Cic in 80% | | Cic in 80% | |
|---|---|---|---|---|---|---|---|---|
| | EtOH | | EtOH/PEG400 | | EtOH | | EtOH/PEG400 | |
| | Mean | | Mean | | Mean | | Mean | |
| Param- | (n = | | (n = | | (n = | | (n = | |
| eters | 6) | Stdev | 6) | Stdev | 5) | Stdev | 5) | Stdev |
| Event | 1.89 | 0.12 | 2.98 | 0.01 | 1.84 | 0.04 | 2.98 | 0.00 |
| dura- | | | | | | | | |
| tion/s | | | | | | | | |
| Dv10/μm | 1.66 | 0.02 | 2.75 | 0.13 | 1.47 | 0.02 | 2.37 | 0.04 |
| Dv50/μm | 3.19 | 0.06 | 5.67 | 0.25 | 2.70 | 0.03 | 4.83 | 0.08 |
| Dv90/μm | 6.18 | 0.26 | 11.87 | 0.96 | 5.01 | 0.06 | 9.82 | 0.30 |

The resulting particle (droplet) size distribution as measured by laser diffraction and reported in Table 9 is summarized in FIG. 12. This data follows the expected trend, where addition of either water, PEG400, or both increases the particle sizes. Here, the 70% ethanol:water mixture has larger particle sizes than the 80% ethanol:water mixture. Similarly, the 70% ethanol:water:PEG400 mixture has larger particle sizes than the 80% ethanol:water:PEG400 mixture.

### Example 9: Comparison of Spray Test Results Using a Solution of Ciclesonide (CIC) in 65%, 70%, and 80% Ethanol:Water Concentrations Via Nebulization by a Soft-Mist Inhaler

A comparison of particle sizes for ciclesonide at different ethanol concentrations in water is summarized in Table 10 and FIG. 13. All measurements were conducted using humidified air.

TABLE 10

| | Cic 65% EtOH | | Cic 70% EtOH | | Cic in 80% EtOH | |
|---|---|---|---|---|---|---|
| | Mean | | Mean | | Mean | |
| Parameters | (n = 6) | Stdev | (n = 6) | Stdev | (n = 5) | Stdev |
| Event | 2.03 | 0.02 | 1.89 | 0.12 | 1.84 | 0.04 |
| duration/s | | | | | | |
| Dv10/μm | 1.80 | 0.06 | 1.66 | 0.02 | 1.47 | 0.02 |
| Dv50/μm | 3.53 | 0.18 | 3.19 | 0.06 | 2.70 | 0.03 |
| Dv90/μm | 6.95 | 0.52 | 6.18 | 0.26 | 5.01 | 0.06 |

As expected, the Dv50 value increases with the addition of additional water.

### Example 10: Comparison of Spray Test Results Using a Solution of Ciclesonide (CIC) in 65%, 70%, and 80% Ethanol:Water:PEG400 Concentrations Via Nebulization by a Soft-Mist Inhaler

A comparison of particle sizes for ciclesonide at different ethanol concentrations in water with PEG400 is summarized in Table 11 and FIG. 14. All measurements were conducted using humidified air.

TABLE 11

| | Cic 65% | | Cic 70% | | Cic in 80% | |
|---|---|---|---|---|---|---|
| | EtOH/PEG400 | | EtOH/PEG400 | | EtOH/PEG400 | |
| | Mean | | Mean | | Mean | |
| Parameters | (n = 6) | Stdev | (n = 6) | Stdev | (n = 5) | Stdev |
| Event | 2.74 | 0.21 | 2.98 | 0.01 | 2.98 | 0.00 |
| duration/s | | | | | | |
| Dv10/μm | 1.77 | 0.05 | 2.75 | 0.13 | 2.37 | 0.04 |
| Dv50/μm | 3.57 | 0.16 | 5.67 | 0.25 | 4.83 | 0.08 |
| Dv90/μm | 7.34 | 0.46 | 11.87 | 0.96 | 9.82 | 0.30 |

Similar to what was observed in Example 7, the addition of PEG400 to the 65%, 70%, 80% ethanol:water mixtures does not result in the expected trend of increased particle sizes with increasing water concentration. Instead, the particle sizes unexpectedly reach a maximum at the 70% ethanol:30% water:200 mg/mL PEG400 concentration.
The following is a list of exemplary numbered embodiments E1 to E20 comprised by the present invention:
E1. A method for the treatment of a respiratory disease, disorder, or condition resulting from a viral infection in a subject, the method comprising the step of administering to said subject a medically active liquid in nebulized form by inhalation,
wherein the medically active liquid comprises an inhalable corticosteroid (ICS) and wherein the medically active liquid is administered in nebulized form using an inhalation device.
E2. The method according to embodiment E1 for the treatment of a pulmonary viral infection in a subject, the method comprising the step of administering to said subject a medically active liquid in nebulized form by inhalation,
wherein the medically active liquid comprises an inhalable corticosteroid (ICS) and wherein the medically active liquid is administered in nebulized form using an inhalation device.
E3. The method according to embodiment E1 or E2, wherein the inhalable corticosteroid is selected from the group consisting of prednisolone, prednisone, butixocort (e.g., butixocort propionate), flunisolide, beclomethasone, triamcinolone, budesonide, fluticasone, mometasone (furoate), ciclesonide, rofleponide, dexamethasone, etiprednol (e.g., etiprednol dichloroacetate), deflazacort, loteprednol, RPR-106541, NS-126 and ST-26.
E4. The method according to any one of embodiments E1 to E3, wherein the inhalable corticosteroid is selected from the group consisting of beclomethasone, budesonide, fluticasone, dexamethasone, mometasone (furoate) and ciclesonide.
E5. The method according to any one of embodiments E1 to E4, wherein the inhalable corticosteroid is ciclesonide.
E6. The method according to any one of embodiments E1 to E5, wherein the medically active liquid comprises two or more inhalable corticosteroids, e.g., ciclesonide in combination with a second inhalable corticosteroid.
E7. The method according to any one of embodiment E1 to E6, wherein the subject is a human or animal.
E8. The method according to any one of embodiments E1 to E7, wherein the inhalable corticosteroid is administered to the lungs of the subject.
E9. The method according to any one of embodiments E1 to E8, wherein the pulmonary viral infection is a lower respiratory tract infection (e.g., a pneumonia).
E10. The method according to any one of embodiments E1 to E9, wherein the pulmonary viral infection is a severe acute respiratory syndrome (SARS).
E11. The method according any one of embodiments E1 to E10, wherein the pulmonary viral infection is a coronavirus infection (e.g., SARS-CoV or SARS-CoV-2 infection).
E12. The method according to embodiment E11, wherein the pulmonary viral infection is a SARS-CoV-2 virus infection.
E13. The method according to embodiment E12, wherein the subject is diagnosed with COVID-19.
E14. The method according to any one of embodiments E1 to E13, wherein the inhalable corticosteroid (ICS) is administered in an amount of about 10 μg (mcg) to about 3,000 μg (mcg) per day.
E15. The method according to any one of embodiments E1 to E14, wherein the inhalable corticosteroid is administered in in 1 to 4 doses per day.
E16. The method according to any one of embodiments E1 to E15, wherein the inhalable corticosteroid is administered for a period of at least 5 days.
E17. The method according to any one of embodiments E1 to E16, wherein the inhalation device used to administer the medically active liquid comprising an inhalable corticosteroid is a hand-held device.
E18. The method according to any one of embodiments E1 to E17, wherein the inhalation device used to administer the medically active liquid comprising an inhalable corticosteroid is a soft-mist-inhaler.
E19. The method according to any one of embodiments E1 to E18, wherein the inhalation device used to administer the medically active liquid comprising an inhalable corticosteroid is a soft-mist-inhaler having at least one impingement-type nozzle.
E20. The method according to any one of embodiments E1 to E19, wherein the inhalation device used to administer the medically active liquid comprising an inhalable corticosteroid is a hand-held inhalation device for delivering a nebulized medically active aerosol for inhalation therapy, comprising
(a) a housing having a user-facing side;
(b) an impingement-type nozzle for generating the nebulized aerosol by collision of at least two liquid jets, the nozzle being firmly affixed to the user-facing side of the housing such as to be immobile relative to the housing;
(c) a fluid reservoir arranged within the housing; and
(d) a pumping unit arranged within the housing, the pumping unit having
   an upstream end that is fluidically connected to the fluid reservoir;
   a downstream end that is fluidically connected to the nozzle;
      wherein the pumping unit is adapted for pumping fluid from the fluid reservoir to the nozzle;
wherein the pumping unit further comprises
   (i) a riser pipe having an upstream end, wherein the riser pipe is
      adapted to function as a piston in the pumping unit, and
      firmly affixed to the user-facing side of the housing such as to be immobile relative to the housing; and
   (ii) a hollow cylinder located upstream of the riser pipe, wherein the upstream end of the riser pipe is inserted in the cylinder such that the cylinder is longitudinally movable on the riser pipe;
   (iii) a lockable means for storing potential energy when locked and for releasing the stored energy when unlocked, the means being arranged outside of, and mechanically coupled to, the cylinder such that unlocking the means results in a propulsive longitudinal movement of the cylinder towards the downstream end of the pumping unit.
      E21. The method according to any one of embodiments E1 to E20, wherein the medically active liquid has a pH from about 3.0 to about 6.0.
      E22. The method according to any one of embodiments E1 to E21, wherein the medically active liquid has a pH from about 4.0 to about 5.0.
      E23. The method according to any one of embodiments E1 to E22, wherein the medically active liquid has a pH from about 4.25 to about 4.75.
      E24. The method according to any one of embodiments E1 to E23, wherein the medically active liquid has a pH of about 4.5.
      E25. The method according to any one of embodiments E1 to E24, wherein the medically active liquid further comprises a polyether compound.
      E26. The method according to embodiment 25, wherein the polyether compound is a polyethylene glycol.
      E27. The method according to embodiment 26, wherein the polyethylene glycol is polyethylene glycol 300 (PEG300) or polyethylene glycol 400 (PEG400).
      E28. The method according to embodiment 27, wherein the polyethylene glycol is polyethylene glycol 300 (PEG300).
      E29. The method according to embodiment 27, wherein the polyethylene glycol is polyethylene glycol 400 (PEG400).
      E30. The method according to any one of embodiments E1 to E29, wherein a Dv50 value of the nebulized medically active aerosol comprising the inhalable corticosteroid is from about 2.0 μm to about 7.0 μm.
      E31. The method according to embodiment E30, wherein a Dv50 value of the nebulized medically active aerosol comprising the inhalable corticosteroid is from about 3.0 μm to about 6.0 μm.
      E32. The method according to embodiment E31, wherein a Dv50 value of the nebulized medically active aerosol comprising the inhalable corticosteroid is from about 4.0 μm to about 6.0 μm at the Dv50.
      E33. The method according to embodiment E32, wherein a Dv50 value of the nebulized medically active aerosol comprising the inhalable corticosteroid is from about 5.0 μm to about 6.0 μm.
      E34. The method according to any one of embodiments E1 to E33, wherein the medically active liquid does not comprise a surfactant.
      E35. The method according to any one of embodiments E1 to E34, wherein the medically active liquid does not comprise a non-ionic surfactant.
      E36. The method according to any one of embodiments E1 to E35, wherein the medically active liquid does not comprise a polysorbate surfactant.
      E37. The method according to any one of embodiments E1 to E36, wherein the medically active liquid does not comprise polysorbate 80.
      E38. The method according to any one of embodiments E1 to E37, wherein the medically active liquid comprises an about 70:30 (w/w %) ethanol:water mixture, an about 80:20 (w/w %) ethanol:water mixture, or an ethanol:water mixture in the range from about 70:30 (w/w %) ethanol:water to about 80:20 (w/w %) ethanol:water.
      E39. The method according to embodiment E38, wherein the medically active liquid comprises an about 70:30 (w/w %) ethanol:water mixture.
      E40. The method according to embodiment E38, wherein the medically active liquid comprises an about 80:20 (w/w %) ethanol:water mixture.
      E41. The method according to any one of embodiments E26 to E29, wherein a concentration of the polyethylene glycol is from about 150 to about 250 mg/mL.
      E42. The method according to any one of embodiments E25 to E29, wherein the concentration of the polyethylene glycol is from about 175 to about 225 mg/mL.
      E43. The method according to any one of embodiments E25 to E29, wherein the concentration of the polyethylene glycol is about 200 mg/mL.

1. A method for the treatment of a respiratory disease, disorder, or condition resulting from a viral infection in a subject, the method comprising the step of administering to said subject a medically active liquid in nebulized form by inhalation,
wherein the medically active liquid comprises an inhalable corticosteroid (ICS), wherein the medically active liquid is administered in nebulized form using an inhalation device, and wherein the inhalation device is a soft-mist-inhaler.

2. The method according to claim 1, wherein the inhalable corticosteroid is selected from the group consisting of beclomethasone, budesonide, fluticasone, mometasone and ciclesonide.

3. The method according to claim 1, wherein the inhalable corticosteroid is ciclesonide.

4. The method according to claim 1, wherein the subject is a human or animal.

5. The method according to claim 1, wherein the viral infection is a severe acute respiratory syndrome (SARS).

6. The method according to claim 1, wherein the viral infection is a coronavirus infection (e.g., SARS-CoV or SARS-CoV-2 infection).

7. The method according to claim 6, wherein the viral infection is a SARS-CoV-2 virus infection.

8. The method according to claim 7, wherein the subject is diagnosed with COVID-19.

9. The method according to claim 1, wherein the medically active liquid has a pH from about 4.0 to about 5.0.

10. The method according to claim 1, wherein the medically active liquid further comprises a polyethylene glycol.

11. The method according to claim 10, wherein the polyethylene glycol is polyethylene glycol 300 or polyethylene glycol 400.

12. The method according to claim 10, wherein the polyethylene glycol has a concentration of about 200 mg/mL.

13. The method according to claim 1, wherein a Dv50 value of the nebulized medically active aerosol comprising the inhalable corticosteroid is from about 2.0 μm to about 7.0 μm.

14. The method according to claim 13, wherein the Dv50 value of the nebulized medically active aerosol comprising the inhalable corticosteroid is from about 3.0 μm to about 6.0 μm.

15. The method according to claim 1, wherein the medically active liquid comprises a 70:30 (w/w %) ethanol:water mixture.

16. The method according to claim 1, wherein the medically active liquid does not comprise a polysorbate surfactant.

17. The method according to claim 16, wherein the medically active liquid does not comprise polysorbate 80.

18. The method according to claim 1, wherein the inhalation device used to administer the medically active liquid comprising an inhalable corticosteroid is a soft-mist-inhaler having at least one impingement-type nozzle.

19. The method according to claim 1, wherein the inhalation device used to administer the medically active liquid comprising an inhalable corticosteroid is a hand-held inhalation device for delivering a nebulized medically active aerosol for inhalation therapy, comprising
(a) a housing having a user-facing side;
(b) an impingement-type nozzle for generating the nebulized aerosol by collision of at least two liquid jets, the nozzle being firmly affixed to the user-facing side of the housing such as to be immobile relative to the housing;
(c) a fluid reservoir arranged within the housing; and
(d) a pumping unit arranged within the housing, the pumping unit having
an upstream end that is fluidically connected to the fluid reservoir;
a downstream end that is fluidically connected to the nozzle;
wherein the pumping unit is adapted for pumping fluid from the fluid reservoir to the nozzle;
wherein the pumping unit further comprises
(i) a riser pipe having an upstream end, wherein the riser pipe is
adapted to function as a piston in the pumping unit, and
firmly affixed to the user-facing side of the housing such as to be immobile relative to the housing; and
(ii) a hollow cylinder located upstream of the riser pipe, wherein the upstream end of the riser pipe is inserted in the cylinder such that the cylinder is longitudinally movable on the riser pipe;
(iii) a lockable means for storing potential energy when locked and for releasing the stored energy when unlocked, the means being arranged outside of, and mechanically coupled to, the cylinder such that unlocking the means results in a propulsive longitudinal movement of the cylinder towards the downstream end of the pumping unit.

20. A kit comprising
a medically active liquid comprising an inhalable corticosteroid (ICS) for the treatment of a respiratory disease, disorder or condition resulting from a viral infection in a subject, wherein the medically active liquid is administered to the subject in nebulized form by inhalation; and
a soft-mist-inhaler.
